(19) United States
(12) Patent Application Publication
(10) Pub. No.: US 2004/0185460 A1
(43) Pub. Date: Sep. 23, 2004

(54) Novel mixed lineage kinase (7) (mlk7) polypeptide polynucleotides encoding the same and methods of use thereof

(76) Inventors: Thelma S Angeles, West Chester, PA (US); John T. Durkin, Ardmore, PA (US); Beverly P Holskin, Philadelphia, PA (US); Sheryl L Meyer, Collegeville, PA (US); Chrysanthe M Spais, West Chester, PA (US)

Correspondence Address: Suzanne E Miller, Woodcock Washburn, 46th Floor, One Liberty Place, Philadelphia, PA, 19103, US

(21) Appl. No.: 10/478,068

(22) PCT Filed: May 23, 2002
(86) PCT No.: PCT/US02/16387

Related U.S. Application Data
(60) Provisional application No. 60/293,381, filed on May 24, 2001.

(51) Int. Cl. C12Q 1/68; C07H 21/04; C12N 9/12; C12N 7/01

(52) U.S. Cl. 435/6; 435/69.1; 435/194; 435/235.1; 435/320.1; 435/325; 536/23.2

(57) ABSTRACT

The present invention provides isolated mixed lineage kinase (7) (MLK) polynucleotides, expression vectors, host cells, isolated polypeptides, methods of producing polypeptides, isolated antibodies, compositions having the foregoing, methods for identifying a compound that binds a polypeptide or polynucleotide, and to methods for identifying a compound that modulates the activity of a poypeptide.

### CROSS-REFERENCE TO RELATED APPLICATIONS

The present application claims priority to U.S. provisional application Serial No. 60/293,381 filed May 24, 2001, which is incorporated herein by reference in its entirety.

### FIELD OF THE INVENTION

The present invention is directed, in part, to a novel mixed lineage kinase 7 (MLK7) polypeptide, polynucleotides encoding the same, methods for identifying compounds that bind to or modulate the activity of the polypeptide, and methods for assaying the enzymatic activity of MLK7.

### BACKGROUND OF THE INVENTION

The MLK family comprises a group of enzymes in which the protein sequence of the kinase domains of the family members show homology with both tyrosine and serine/threonine protein kinases. In particular, this region contains many amino acids highly conserved among tyrosine kinases, but the homology to serine/threonine protein kinases in the catalytic domain, subdomains VIb and VIII, is consistent with the known serine/threonine kinase activity of MLK3 (Gallo et al., J. Biol. Chem., 1994,269, 15092-15100). In addition, all family members contain a leucine zipper, which is an α-helical structure characterized by a periodic repeat of leucine or isoleucine every seventh amino acid for a stretch of at least 22 amino acids to form a parallel coiled-coil structure (Landschulutz et al., Science, 1988, 240, 1759-1764). These kinases comprise a portion of very complex kinase cascades such as, for example, the stress-signaling cascade, which involves modulation of, inter alia, c-Jun N-terminal kinase (JNK), which in turn modulates, inter alia, transcription factors including c-Jun, ATF2 and ELK1. JNK is described in U.S. Pat. Nos. 5,534,426, 5,593,884, 5,605,808, and in international publication WO 95/03324.

The MLK family consists of 6 reported family members: 1) mixed lineage kinase 1 (MLK1); 2) mixed lineage kinase 2 (MLK2); 3) mixed lineage kinase 3 (MLK3); 4) dual leucine zipper bearing kinase (DLK); 5) leucine zipper bearing kinase (LZK), and 6) MLK-like mitogen activated protein triple kinase/mixed lineage kinase 6 (MLTK/MLK6). A partial sequence has been reported for MLK1 (Dorow et al., Eur. J. Biochem., 1993, 213, 701-710). Mixed lineage kinases 1, 2 and 3 have, in addition to the features described above, a src-homology-3 (SH3) domain N-terminal to the kinase domain (Dorow et al., J. Protein Chem., 1994, 13, 458-460; Dorow et al., Eur. J. Biochem., 1995, 234, 492-500; and Ing et al., Oncogene, 1994, 9, 1745-1750). Their leucine zipper region is just C-terminal to the kinase domain and is comprised of 2 repeats of 4 alpha helical regions separated by a small spacer region, termed a dual leucine zipper. Deletion analysis suggests a functional role of this domain in homodimerization (Leung et al., J. Biol. Chem., 1998, 273, 32408-32415). This domain is followed by a small region comprised of a preponderance of basic amino acids similar to nuclear localization signals from several proteins (Dorow et al., Eur. J. Biochem., 1995, 234, 492-500). Immediately adjacent to that is a Cdc42/Rac interactive binding (CRIB) domain (Burbelo et al., J. Biol. Chem., 1995, 270, 29071-29074). The C-terminus has generally a low level of complexity, where 3 amino acids (proline, serine and glycine) comprise up to 41% (MLK3) of the primary amino acid sequence. MLK2 is also known as MST (Katoh et al., Oncogene, 1995, 10, 1447-145 1). MLK3 is also known as scr-homology 3 (SH3) domain-containing proline-rich kinase (SPRK) (Gallo et al., J. Biol. Chem., 1994, 269, 15092-15100) and protein tyrosine kinase 1 (PTK1) (Ezoe et al., Oncogene, 1994, 9, 935-938).

DLK and LZK share the same general features, but do not have the SH3 and CRIB domains and their spacer region in the dual leucine zipper is about 18 amino acids longer than in MLKs 1-3 (Holtzman et al., J. Biol. Chem., 1994, 269, 30808-30817; Sakuma et al., J. Biol. Chem., 1997, 272, 28622-28629). DLK is also known as leucine-zipper protein kinase (ZPK) (Reddy et al., Biochem. Biophys. Res. Commun., 1994, 202, 613-620) and MAPK-upstream kinase (MUK) (Hirai et al., Oncogene, 1996, 12, 641-650).

MLTK/MLK6 is even more divergent. They share a kinase domain and putative leucine zipper region, but like the DLK/LZK group, there is no SH3 binding domain or CRIB domain, nor is there a basic amino acid region. Rather, the two cDNA sequences diverge after the putative leucine zipper region, resulting in two different isoforms, alpha and beta. Furthermore, unlike the other family members containing the “dual” 4+4 repeats, the putative leucine zipper region contains six contiguous alpha-helical repeats. In addition, unlike other family members, the kinase domain is essentially at the N-terminus (Gotoh et al., J. Biol. Chem., 2001, 276, 4276-4286). MLTKα/MLK6α is also known as leucine-zipper sterile alpha motif kinase (ZAK) (Liu et al., Biochem. Biophys. Res. Commun., 2000, 274, 811-816).

Members of the MLK family are also described in, for example, U.S. Pat. No. 5,676,945; U.S. Pat. No. 5,554,523; international publication WO 93/15201; Canadian Patent 2,148,898; Diener et al., Proc. Natl. Acad. Sci. USA, 1997, 94, 9687-9692; DeAizpurua et al., J. Biol. Chem., 1997, 272, 16364-16373;Tung et al., Oncogene, 1997, 14, 653-659; Sells et al., Trends in Cell Biol., 1997, 7, 161-167; Mata et al., J. Biol. Chem., 1996, 271, 16888-16896; Hirai et al., J. Biol. Chem., 1997, 272, 15167-15173; Fan et al., J. Biol. Chem., 1996, 271, 24788-24793; Blouin et al., DNA and Cell Biol., 1996, 15, 631-642; Pombo et al., Nature, 1995, 377, 750-754; Kiefer et al., EMBO J., 1996, 15, 7013-7025; Hu et al., Genes & Dev., 1996, 10, 2251-2264; and Su et al., EMBO J., 1997, 16, 1279-1290. Portions of the kinase domain for human kinase homologs have also been reported in U.S. Pat. No. 5,817,479. In addition, nucleotide sequences for a mixed lineage kinase mRNA have been reported in the Genbank database (see, Accession Numbers AF238255 and AF251441).

Due to the inadequacies of screening compounds that modulate members of the stress signaling cascade and promote either cell death or cell survival, there continues to be a need for new, selective methods of screening compounds. In addition, there continues to be a need for screening assays for therapeutics which may be useful in treating inflammation and neurodegenerative disorders. The present invention is directed to these, as well as other, important ends. In particular, the present invention describes an additional member of the MLK family, a seventh family member, termed herein as MLK7, identified from human genomic sequences that encode a kinase domain 73-76% homologous to the MLK 1-3 subfamily. MLK7 contains an SH3 domain N-terminal to the kinase domain. MLK7 also contains a dual leucine zipper C-terminal to the kinase domain, followed by a small region comprised of a preponderance of basic amino acids similar to nuclear localization signals from several proteins. This was used to clone a cDNA that encodes a functional kinase protein. The novel polypeptide of the present invention can be used, for example, to phosphorylate MAP kinase kinase 4 (MKK4) and MKK7, and play a role in JNK stress-activated kinase pathway activation. Thus, the polypeptide of the present invention can be used, for example, to identify inhibitors of the same and such inhibitors can be used to promote neuronal cell survival. Compounds that decrease activity of the MLK polypeptides are useful for, for example, promoting cell survival, treating neurodegenerative disorders, and treating inflammation.

### SUMMARY OF THE INVENTION

The present invention provides a novel MLK7 polypeptide and polynucleotides encoding the same.

In particular, the present invention is directed to isolated polynucleotides comprising a nucleotide sequence set forth in SEQ ID NO:1, polynucleotides homologous to SEQ ID NO:1, polynucleotides that encode a polypeptide comprising SEQ ID NO:2, and polynucleotides that encode a polypeptide comprising an amino acid sequence homologous to SEQ ID NO:2.

The present invention is also directed to polynucleotides comprising a nucleotide sequence complementary to at least a portion of SEQ ID NO:1.

The present invention is also directed to expression vectors comprising the polynucleotides of the invention and host cells comprising the same.

The present invention is also directed to isolated polypeptides encoded by the polynucleotides of the invention and to methods of producing polypeptides comprising SEQ ID NO:2 and polypeptides homologous thereto, by introducing a recombinant expression vector comprising a polynucleotide of the invention into a compatible host cell, growing the host cell under conditions for expression of the polypeptide, and recovering the polypeptide.

The present invention is also directed to isolated antibodies that bind to an epitope on a polypeptide of the invention.

The present invention is also directed to compositions comprising a polynucleotide, expression vector, polypeptide, or antibody and a carrier or diluent, and to kits comprising a polynucleotide, expression vector, polypeptide, or antibody.

The present invention is also directed to methods for identifying a compound that binds a polypeptide of the invention by contacting the polypeptide with the compound and determining whether the compound binds to the polypeptide.

The present invention is also directed to methods for identifying a compound that binds a polynucleotide of the invention by contacting the polynucleotide with the compound and determining whether the compound binds the polynucleotide.

The present invention is also directed to methods for identifying a compound that modulates the activity of a polypeptide of the invention by contacting the polypeptide with the compound and determining whether the polypeptide activity has been modulated.

The present invention is also directed to compounds identified by the identification methods of the invention.

### BRIEF DESCRIPTION OF THE FIGURES

FIG. 1 shows a representative kinase subdomain alignment and functional domains for MLK1 and MLK3 from the world wide web of the internet at sdsc.edu/Kinases/pkr/pk_catalytic/pk_hanks_seq_align_long.html#OPK-XI. Other proteins aligned by DNAstar program using Clustal method with PAM250 residue weight table. Amino acids in bold are highly conserved amino acids in protein tyrosine kinases; amino acids in bold italics are conserved in protein serine-threonine kinases; amino acids in bold italics underlined are highly conserved in both (Hanks et al., Meth. in Enzymol., 1991, 200, 38-62). Conserved amino acids for tyrosine kinases altered in these proteins are shown in bold above the sequence (capital letters for conserved, small letters for preferred).

FIG. 2 depicts a representative phylogenetic tree of the kinase domains in the human mixed lineage kinase family. This analysis was performed using the MegAlign feature of the DNAStar program (Clustal method with PAM250 residue weight table). The proteins are, from top to bottom, MLK3, MLK7, MLK1, MLK2, DLK, LZK and MLTK/MLK6. This alignment organizes the seven proteins into three subfamilies: the MLK1-3 subfamily (MLKs 1, 2, 3 and 7), the DLK subfamily (DLK, LZK) and the MLTK/MLK6 subfamily.

FIG. 3 shows a representative domain structure of MLK7 polypeptide and the predicted molecular weight (MW) of the polypeptide.

FIG. 4 shows the sequence of the putative leucine zipper region that follows the kinase domain. The leucine/isoleucine residues spaced every seventh amino acid (one alpha-helical turn) are shown in bold and are underlined. The seventh amino acid preceding and following this region begin and end the shown sequence to demonstrate that this pattern does not continue.

FIG. 5 shows that overexpression of MLK7 increases JNK activity in CHO cell lysates, and that six-hour treatment with CEP11004 partially inhibits JNK activity driven by MLK7.

FIG. 6 illustrates the phosphorylation of myelin basic protein by baculoviral GST-MLK7_{KD} (open circles) and GST-MLK7_{KD/LZ} (filled-in circles) using a radioactive multiscreen trichloroacetic acid precipitation assay. The entire data set containing the positive enzyme control GST-MLK3_{KD} (filled-in diamonds) is shown in the inset.

FIG. 7 shows the phosphorylation of (A) GST-MKK4(K113A) and (B) GST-MKK7(K149A) by baculoviral GST-MLK7_{KD} (open circles) and GST-MLK7_{KD/LZ} (filled-in circles) using an ELISA-based assay with time-resolved fluorescence readout. GST-MLK3_{KD} (filled-in diamonds) was used as the positive enzyme control.

FIG. 8 demonstrates the in vitro activation of the JNK pathway by baculoviral GST-MLK7_{KD} and GST-MLK7_{KD/LZ}. Inactivated forms of MKK4 and MKK7 were separately phosphorylated by baculoviral GST-MLK7_{KD}. Subsequently, these two MKK proteins were activated and proceeded to catalyze the phosphorylation of GST-JNK1β1(K55A) as probed by a phospho-specific JNK antibody. Similar results were observed for GST-MLK7_{KD/LZ} and GST-MLK3_{KD} (enzyme control), albeit to different efficiencies. Inset shows the entire data set, including controls.

### DESCRIPTION OF PREFERRED EMBODIMENTS

The present invention provides purified and isolated polynucleotides (e.g., DNA sequences and RNA transcripts, chimeric RNA/DNA molecules, both sense and complementary antisense strands, both single- and double-stranded, including splice variants thereof) encoding a human MLK polypeptide referred to herein as “MLK7.” DNA polynucleotides of the invention include genomic DNA, cDNA, and DNA that has been chemically synthesized.

As used herein, “activity” means a variety of measurable indicia suggesting or revealing binding, either direct or indirect; affecting a response, such as having a measurable affect in response to some exposure or stimulus, including, for example, the affinity of a compound for directly binding a polypeptide or polynucleotide of the invention, or, for example, measurement of amounts of upstream or downstream proteins (e.g. JNK) or activity thereof, or other similar functions after a stimulus or event.

As used herein, “antibody” means complete, intact antibodies, and Fab, Fab′, F(ab)₂, and other fragments thereof. Complete, intact antibodies include monoclonal antibodies, chimeric antibodies, and humanized antibodies.

As used herein, “binding” means the physical or chemical interaction between two polypeptides or polynucleotides or compounds or associated proteins or compounds or combinations thereof. Binding includes, but is not limited to, ionic, non-ionic, Hydrogen bonds, Van der Waals, hydrophobic interactions, etc. Binding can be either direct or indirect through or due to the effects of another protein or compound.

As used herein, “compound” means any chemical or molecule including, but not limited to, small molecules, peptides, proteins or polypeptides, sugars, nucleotides, or nucleic acids, and the like. The compounds can be natural or synthetic.

As used herein, “contacting” means bringing together, either directly or indirectly, a compound into physical proximity to a polypeptide or polynucleotide of the invention. The polypeptide or polynucleotide can be in any number of buffers, salts, solutions, etc. Contacting includes, but is not limited to, placing the compound into a beaker, microtiter plate, cell culture flask, or a microarray, or the like, which contains the MLK7 polypeptide or fragment thereof, polynucleotide encoding the same, or antibody binding to the same.

As used herein, “homologous nucleotide sequence,” or “homologous amino acid sequence,” or variations thereof, means sequences characterized by a homology, at the nucleotide level or amino acid level, of at least a specified percentage. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. A homologous nucleotide sequence is not, however, identical to the nucleotide sequences encoding other known MLK polypeptides. Homologous amino acid sequences include those amino acid sequences that encode conservative amino acid substitutions, as well as polypeptides having kinase activity. A homologous amino acid sequence is not, however, identical to the amino acid sequences encoding other known MLK polypeptides. Percent homology can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489, which is incorporated herein by reference in its entirety).

As used herein, “modulates” or “modifies” means an increase or decrease in the amount, quality, or effect of a particular activity or polypeptide.

As used herein, “treating” means to having a therapeutic effect and at least partially alleviating or abrogating an abnormal condition in an organism.

As used herein, “therapeutic effect” means inhibition or activation of factors causing or contributing to an abnormal condition. A therapeutic effect alleviates, at least to some extent, one or more of the symptoms of the abnormal condition. In regard to treatment of abnormal conditions, a therapeutic effect can refer to one or more of the following: i) an increase in the proliferation, growth, and/or differentiation of cells; ii) inhibition (i.e., slowing or stopping) of cell death; iii) inhibition of degeneration; iv) relieving to some extent one or more of the symptoms associated with the abnormal condition; and v) enhancing the function of the affected population of cells.

As used herein, “abnormal condition” means a function in cells or tissues of an organism that deviates from their normal functions in that organism. An abnormal condition can be any condition that is not desired by an individual. An abnormal condition can relate to cell proliferation, cell differentiation, cell signaling, or cell survival. Abnormal differentiation conditions include, but are not limited to, neurodegenerative or inflammatory disorders. Abnormal cell survival conditions can also relate to conditions in which programmed cell death (apoptosis) pathways are activated or abrogated.

As used herein, “administering” means a method of incorporating a compound into a cell or tissue of an organism. For cells within an organism, administration techniques include, but are not limited to, oral, intramuscular, intraperitoneal, intradermal, subcutaneous, intravenous, intraarterially, intraoccularly, intraventricularly, and transdermally, as well as by inhalation or suppository. For cells outside an organism, administration techniques include, but are not limited to, cell microinjection, transformation, and carrier techniques.

As used herein, “organism” means a mammal such as a mouse, rat, rabbit, guinea pig or goat, more preferably a primate such as a monkey or ape, and most preferably a human.

As used herein, “stringent hybridization conditions” or “stringent conditions” means conditions under which a probe, primer, or oligonucleotide will hybridize to its target sequence, but to no other sequences. Stringent conditions are sequence-dependent and will be different in different circumstances. Generally, stringent conditions are selected to be about 5 C lower than the thermal melting point (Tm) for the specific sequence at a defined ionic strength and pH. Target sequences are generally present in excess. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 C for short probes, primers or oligonucleotides (e.g. 10 to 50 nucleotides) and at least about 60 C for longer probes, primers or oligonucleotides. Stringent conditions can also be achieved with the addition of destabilizing agents, such as formamide.

The polynucleotides of the invention can include genomic DNA which comprises the protein coding region for a polypeptide of the invention and is also intended to include allelic variants or splice variants thereof. Splice variants of the invention are encoded by the same original genomic DNA sequences but arise from distinct mRNA transcripts. Allelic variants are modified forms of a wild-type gene sequence, the modification resulting from recombination during chromosomal segregation or exposure to conditions which give rise to genetic mutation. Allelic variants, like wild-type genes, are naturally occurring sequences, as opposed to non-naturally occurring variants which arise from in vitro manipulation.

In the present invention, a polynucleotide encoding MLK7 polypeptide is set forth in SEQ ID NO:1. A preferred polypeptide of the invention comprises a double stranded molecule. Also preferred are other polynucleotides encoding particular MLK7 polypeptides of the invention which differ in sequence from the particular polynucleotide set forth in SEQ ID NO:1 by virtue of the well-known degeneracy of the universal nuclear genetic code.

The invention is further directed to additional species homologs, preferably mammalian, of the human MLK7 polynucleotides. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with human polynucleotides of the invention. Generally, percent sequence “homology” with respect to polynucleotides of the invention can be calculated as the percentage of nucleotide bases in the candidate sequence that are identical to nucleotides in the MLK7 sequence set forth in a particular polynucleotide sequence, after aligning the sequences and introducing gaps, if necessary, to achieve the maximum percent sequence identity.

Examples of related polynucleotides include human and non-human genomic sequences, including allelic variants, as well as polynucleotides encoding polypeptides homologous to MLK7 polypeptides and structurally related polypeptides sharing one or more biological, immunological, and/or physical properties of MLK7 polypeptides. Non-human species genes encoding proteins homologous to MLK7 polypeptides can also be identified by Southern and/or PCR analysis and are useful in animal models for MLK polypeptide disorders. Polynucleotides of the invention are also useful in hybridization assays to detect the capacity of cells to express MLK7 polypeptides. Polynucleotides of the invention can also provide a basis for diagnostic methods useful for identifying a genetic alteration(s) in an MLK7 locus that underlies a disease state or states, such information being useful both for diagnosis and for selection of therapeutic strategies.

The disclosure herein of a full-length polynucleotide encoding a MLK7 polypeptide makes readily available to the worker of ordinary skill in the art every possible fragment of the full length polynucleotide. MLK7 polynucleotides, therefore, encompass fragments comprising at least 14, and preferably at least 16, 18, 20, 25, 50, 75, 100, 150, 200, 250, 400, 500, 750, 1000, 1250, 1500, 1750, 2000, 2250, 2500, 2750, or 3000 consecutive nucleotides of a polynucleotide encoding MLK7. Preferably, fragment polynucleotides of the invention comprise sequences unique to the MLK7-encoding polynucleotide sequence, and therefore hybridize under highly stringent or moderately stringent conditions only (i.e., “specifically”) to polynucleotides encoding MLK7 (or fragments thereof). Polynucleotide fragments of genomic sequences of the invention comprise not only sequences unique to the coding region, but also include fragments of the full-length sequence derived from introns, regulatory regions, and/or other non-translated sequences. Polynucleotides of the invention can be labeled in a manner that permits their detection, including radioactive, fluorescent, and enzymatic labeling.

Fragment polynucleotides are particularly useful as probes for detection of full-length or fragment MLK7 polynucleotides. One or more polynucleotides can be included in kits that are used to detect the presence of a polynucleotide encoding MLK7, or used to detect variations in a polynucleotide sequence encoding MLK7.

The invention is also directed to polynucleotides encoding MLK7 polypeptides that hybridize under moderately stringent or high stringency conditions to the non-coding strand, or complement, of the polynucleotide in SEQ ID NO:1. Exemplary highly stringent hybridization conditions are as follows: hybridization at 42 C in a hybridization solution comprising 50% formarnmide, 1% SDS, 1 M NaCl, 10% Dextran sulfate, and washing twice for 30 minutes at 60 C in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley& Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51, which is incorporated herein by reference in its entirety.

Recombinant expression constructs such as plasmids and viral DNA vectors incorporating polynucleotides of the invention are also provided. Preferably, MLK7-encoding polynucleotides are operatively linked to an endogenous or exogenous promoters, enhancers, operators, or regulatory element binding sites, or any combination thereof. Expression constructs of the invention can also include sequences encoding one or more selectable markers that permit identification of host cells bearing the construct. Expression constructs can also include sequences that facilitate, and preferably promote, homologous recombination in a host cell. Preferred constructs of the invention also include sequences necessary for replication in a host cell. Expression constructs are preferably utilized for production of an encoded protein, but can also be utilized to amplify a MLK7-encoding polynucleotide sequence.

In another embodiment of the invention, host cells are provided, including prokaryotic and eukaryotic cells, comprising a polynucleotide of the invention (or vector of the invention) in a manner which permits expression of the encoded MLK7 polypeptide. Polynucleotides of the invention can be introduced into the host cell as part of a plasmid, or as linear DNA comprising an isolated protein coding region or a viral vector. Methods for introducing DNA into a host cell are well known and routinely practiced in the art and include transformation, transfection, electroporation, nuclear injection, or fusion with carriers such as liposomes, micelles, ghost cells, and protoplasts. Expression systems of the invention include bacterial, yeast, fungal, plant, insect, invertebrate, vertebrate, and mammalian cells systems.

Host cells of the invention can provide immunogens based on the MLK7 polypeptide for development of antibodies that are specifically immunoreactive with MLK7. Host cells of the invention are also useful in methods for the large-scale production of MLK7 polypeptide wherein the cells are grown in a suitable culture medium and the desired polypeptide product is isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

The present invention is also directed to antisense polynucleotides that recognize and hybridize to polynucleotides encoding MLK7. Full-length and fragment antisense polynucleotides are provided. Fragment antisense molecules of the invention include those that specifically recognize and hybridize to MLK7 RNA or DNA. Antisense polynucleotides are particularly relevant to regulating expression of MLK7 by those cells expressing MLK7 mRNA. Antisense polynucleotides (preferably 10 to 20 base-pair oligonucleotides) capable of specifically binding to MLK7 expression control sequences or MLK7 RNA are introduced into cells (e.g., by a viral vector or colloidal dispersion system such as a liposome). Phosphorothioate and methylphosphonate antisense oligonucleotides are specifically contemplated for therapeutic use by the invention. Suppression of MLK7 expression at either the transcriptional or translational level is useful to generate cellular or animal models for diseases/conditions characterized by aberrant MLK7 expression.

The invention also provides purified and isolated mammalian MLK7 polypeptides encoded by a polynucleotide of the invention. Preferably, the MLK7 polypeptide comprises the amino acid sequence set out in SEQ ID NO:2. The present invention is also directed to polypeptides that have at least 99%, at least 95%, at least 90%, at least 85%, at least 80%, at least 75%, at least 70%, at least 65%, at least 60%, at least 55% or at least 50% identity and/or homology to the preferred polypeptide of the invention. Percent amino acid sequence “identity” with respect to the preferred polypeptide of the invention is defined herein as the percentage of amino acid residues in the candidate sequence that are identical with the residues in the MLK7 sequence after aligning both sequences and introducing gaps, if necessary, to achieve the maximum percent sequence identity, and not considering any conservative substitutions as part of the sequence identity. Percent sequence “homology” with respect to the preferred polypeptide of the invention is defined herein as the percentage of amino acid residues in the candidate sequence that are identical with the residues in the MLK7 sequence after aligning the sequences and introducing gaps, if necessary, to achieve the maximum percent sequence identity, and also considering any conservative substitutions as part of the sequence identity.

The polypeptide of the invention can be isolated from natural cell sources or can be chemically synthesized, but is preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of MLK7 polypeptides are also provided.

The present invention is also directed to MLK7 polypeptide variants or analogs. In one example, insertion variants are provided wherein one or more amino acid residues supplement an MLK7 amino acid sequence. Insertions can be located at either or both termini of the protein, or can be positioned within internal regions of the MLK7 amino acid sequence. Insertion variants with additional residues at either or both termini can include, for example, fusion proteins and proteins including amino acid tags or labels. Insertion variants include MLK7 polypeptides wherein one or more amino acid residues are added to a MLK7 acid sequence, or to a biologically active fragment thereof. Variant products of the invention also include mature MLK7 polypeptides wherein leader or signal sequences are removed, with additional amino terminal residues. The additional amino terminal residues can be derived from another protein, or can include one or more residues that are not identifiable as being derived from specific proteins.

The present invention also provides MLK7 variants having additional amino acid residues that result from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants that result from expression in other vector systems are also contemplated. Insertional variants also include fusion proteins wherein the amino terminus and/or the carboxy terminus of MLK7 is/are fused to another polypeptide. In addition, other fusion proteins comprising MLK7 or fragments thereof are contemplated by the invention. Numerous fusion partner proteins are well known to the skilled artisan.

In another aspect, the invention provides deletion variants wherein one or more amino acid residues in a MLK7 polypeptide are removed. Deletions can be effected at one or both termini of the MLK7 polypeptide, or with removal of one or more non-terminal amino acid residues of MLK7. Deletion variants, therefore, include all fragments of an MLK7 polypeptide.

The invention also embraces polypeptide fragments of SEQ ID NO:2 wherein the fragments maintain biological (e.g., ligand binding and/or kinase activity) or immunological properties of an MLK7 polypeptide. Fragments comprising at least 5, 10, 15, 20, 25, 30, 35, 40, 50, 75, 100, 150, 200, 250, 300, 350, 400, 450, 500, 550, 600, 650, 700, 750, 800, 850, 900, 950, or 1000 consecutive amino acids of any of the polypeptides described herein are contemplated by the present invention. Preferred polypeptide fragments have antigenic properties unique to, or specific for, human MLK7 and its allelic and species homologs. Fragments of the invention having the desired biological and immunological properties can be prepared by any of the methods well known and routinely practiced in the art. Additional preferred fragments include the kinase domain of MLK7 (MLK7_{KD}), the leucine zipper region of MLK7 (MLK7_{LZ}), and the kinase domain linked to the leucine zipper region of MLK7 (MLK7_{KD/LZ}).

In another aspect of the present invention, substitution variants of MLK7 polypeptides are provided. Substitution variants include those polypeptides wherein one or more amino acid residues of a MLK7 polypeptide are removed and replaced with alternative residues. In one aspect, the substitutions are conservative in nature. The present invention, however, also includes substitutions that are also non-conservative. Conservative substitutions for this purpose can be defined as set out below. Variant polypeptides include those wherein conservative substitutions have been introduced by modification of polynucleotides encoding polypeptides of the invention. A conservative substitution is recognized in the art as a substitution of one amino acid for another amino acid that has similar properties. Exemplary conservative substitutions include, but are not limited to, non-polar (Gly, Ala, Pro, Ile, Leu, and Val), polar-uncharged (Cys, Ser, Thr, Met, Asn, and Gln), polar-charged (Asp, Glu, Lys, and Arg) aromatic (His, Phe, Trp, and Tyr), and other (Asn, Gln, Asp and Glu). Alternatively, conservative amino acids can be grouped as described in Lehninger, (Biochemistry, Second Edition; Worth Publishers, Inc. NY, N.Y. (1975), pp.71-77) wherein conservative substitutions include, but are not limited to, non-polar (hydrophobic) 1) aliphatic: Ala, Leu, Ile, Val, and Pro; 2) aromatic: Phe and Trp 3) sulfur-containing: Met; 4) borderline: Gly; or uncharged-polar 1) hydroxyl: Ser, Thr, and Tyr; 2) amides: Asn and Gln; 3) sulfhydryl: Cys; 4) borderline: Gly; or positively charged (basic): Lys, Arg, and His; or negatively charged (acidic): Asp and Glu. As still another alternative, exemplary conservative substitutions include, but are not limited to, Ala (Val, Leu, Ile), Arg (Lys, Gln, Asn), Asn (Gln, His, Lys, Arg), Asp (Glu), Cys (Scr), Gln (Asn), Glu (Asp), His (Asn, Gln, Lys, Arg), Ile (Leu, Val, Met, Ala, Phe), Leu (Ile, Val, Met, Ala, Phe), Lys (Arg, Gln, Asn), Met (Leu, Phe, Ile), Phe (Leu, Val, Ile, Ala), Pro (Gly), Ser (Thr), Thr (Ser), Trp (Tyr), Tyr (Trp, Phe, Thr, Ser), and Val (Ile, Leu, Met, Phe, Ala). It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties.

In a related embodiment, the present invention provides compositions comprising purified polypeptides of the invention. Preferred compositions comprise, in addition to the polypeptide of the invention, a liquid, semisolid, or solid diluent that serves as a vehicle, excipient, carrier or medium. The compositions can also comprise, in addition to the polypeptide of the invention, a pharmaceutically acceptable (i.e., sterile and non-toxic) liquid, semisolid, or solid diluent that serves as a vehicle, excipient, carrier or medium. Any diluent known in the art can be used including, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter.

A polynucleotide comprising any of the MLK7 nucleotide sequences described above can be synthesized by PCR, with the PCR oligonucleotide primers produced from the nucleotide sequences provided herein. See U.S. Pat. Nos. 4,683,195 and 4,683,202. In addition, numerous cloning and in vitro amplification methodologies are well known to those skilled in the art. Examples of these techniques are found in, for example, Berger et al., Guide to Molecular Cloning Techniques, Methods in Enzymology, 152, Academic Press, Inc., San Diego, Calif. (Berger), which is incorporated herein by reference in its entirety.

The polynucleotides of the present invention, and fragments derived therefrom, are useful for screening for restriction fragment length polymorphism (RFLP) associated with particular disorders, as well as for genetic mapping.

Antisense oligonucleotides, or fragments of a nucleotide sequence set forth in SEQ ID NO:1, or sequences complementary or homologous thereto, derived from the nucleotide sequences of the present invention encoding MLK7 are useful as diagnostic tools for probing gene expression in various tissues. For example, tissue can be probed in situ with oligonucleotide probes carrying detectable groups by conventional autoradiography techniques to determine native expression of the polynucleotide or pathological conditions relating thereto. Antisense oligonucleotides are preferably directed to regulatory regions of a nucleotide sequence set forth in SEQ ID NO:1, or mRNA corresponding thereto, including, but not limited to, the initiation codon, TATA box, enhancer sequences, other regulatory sequences, and the like.

Automated sequencing methods can be used to obtain or verify the nucleotide sequence of an MLK7 polynucleotide. The MLK7 polynucleotide sequences of the present invention are believed to be 100% accurate. However, as is known in the art, nucleotide sequence obtained by automated methods can contain some errors. Nucleotide sequences determined by automation are typically at least about 90%, more typically at least about 95% to at least about 99.9% identical to the actual nucleotide sequence of a given nucleic acid molecule. The actual sequence can be more precisely determined using manual sequencing methods, which are well known in the art.

Another aspect of the present invention is directed to vectors, or recombinant expression vectors, comprising any of the nucleic acid molecules described above. Vectors are used herein either to amplify DNA or RNA encoding MLK7 and/or to express DNA which encodes MLK7. Preferred vectors include, but are not limited to, plasmids, phages, cosmids, episomes, viral particles or viruses, and integratable DNA fragments. Preferred viral particles include, but are not limited to, adenoviruses, baculoviruses, parvoviruses, herpesviruses, poxviruses, adeno-associated viruses, Semliki Forest viruses, vaccinia viruses, and retroviruses. Preferred expression vectors include, but are not limited to, pcDNA3 (Invitrogen) and pSVL (Pharmacia Biotech). Other expression vectors include, but are not limited to, pSPORT vectors, pGEM vectors (Promega), pPROEX vectors (LTI, Bethesda, Md.), Bluescript vectors (Stratagene), pQE vectors (Qiagen), pSE420 (Invitrogen), and pYES2 (Invitrogen).

Preferred expression vectors are replicable polynucleotide constructs in which a polynucleotide sequence encoding MLK7 is operably linked or connected to suitable control sequences capable of effecting the expression of an MLK7 in a suitable host. Generally, control sequences include a transcriptional promoter, an optional operator sequence to control transcription, a sequence encoding suitable mRNA ribosomal binding, and sequences which control the termination of transcription and translation. Preferred vectors preferably contain a promoter that is recognized by the host organism. The promoter sequences of the present invention can be prokaryotic, eukaryotic or viral. Examples of suitable prokaryotic sequences include the PR and PL promoters of bacteriophage lambda (The bacteriophage Lambda, Hershey, A. D., Ed., Cold Spring Harbor Press, Cold Spring Harbor, N.Y. (1973), which is incorporated herein by reference in its entirety; Lambda II, Hendrix, R. W., Ed., Cold Spring Harbor Press, Cold Spring Harbor, N.Y. (1980), which is incorporated herein by reference in its entirety); the trp, recA, heat shock, and lacZ promoters of E. coli and the SV40 early promoter (Benoist et al., Nature, 1981, 290, 304-310, which is incorporated herein by reference in its entirety). Additional promoters include, but are not limited to, mouse mammary tumor virus, long terminal repeat of human immunodeficiency virus, maloney virus, cytomegalovirus immediate early promoter, Epstein Barr virus, rous sarcoma virus, human actin, human myosin, human hemoglobin, human muscle creatine, and human metalothionein.

Additional regulatory sequences can also be included in preferred vectors. Preferred examples of suitable regulatory sequences are represented by the Shine-Dalgarno of the replicase gene of the phage MS-2 and of the gene cII of bacteriophage lambda. The Shine-Dalgarno sequence can be directly followed by DNA encoding MLK7 and result in the expression of the mature MLK7. Moreover; suitable expression vectors can include an appropriate marker that allows the screening of the transformed host cells. An origin of replication can also be provided either by construction of the vector to include an exogenous origin or can be provided by the host cell chromosomal replication mechanism. If the vector is integrated into the host cell chromosome, the latter may be sufficient. Alternatively, rather than using vectors which contain viral origins of replication, one skilled in the art can transform mammalian cells by the method of co-transformation with a selectable marker and MLK7 DNA. An example of a suitable marker is dihydrofolate reductase (DHFR) or thymidine kinase (tk) (see, U.S. Pat. No. 4,399,216). Methods for construction of mammalian expression vectors are disclosed in, for example, Okayama et al., Mol. Cell. Biol., 1983, 3, 280, Cosman et al., Mol. Immunol., 1986, 23, 935, Cosman et al., Nature, 1984, 312, 768, EP-A-0367566, and international publication WO 91/18982, each of which is incorporated herein by reference in its entirety.

Another aspect of the present invention is directed to transformed host cells having an expression vector comprising any of the nucleic acid molecules described above. Expression of the nucleotide sequence occurs when the expression vector is introduced into an appropriate host cell. Suitable host cells for expression of the polypeptides of the invention include, but are not limited to, prokaryotes, yeast, and eukaryotes. If a prokaryotic expression vector is employed, then the appropriate host cell can be any prokaryotic cell capable of expressing the cloned sequences. Suitable prokaryotic cells include, but are not limited to, bacteria of the genera Escherichia, Bacillus, Salmonella, Pseudomonas, Streptomyces, and Staphylococcus.

If a eukaryotic expression vector is employed, then the appropriate host cell can be any eukaryotic cell capable of expressing the cloned sequence. Preferably, eukaryotic cells are cells of higher eukaryotes. Suitable eukaryotic cells include, but are not limited to, non-human mammalian tissue culture cells and human tissue culture cells. Preferred host cells include, but are not limited to, insect cells, HeLa cells, Chinese hamster ovary cells (CHO cells), African green monkey kidney cells (COS cells), human 293 cells, and murine 3T3 fibroblasts. Propagation of such cells in cell culture has become a routine procedure (see, Tissue Culture, Academic Press, Kruse and Patterson, eds. (1973), which is incorporated herein by reference in its entirety).

In addition, a yeast host may be employed as a host cell. Preferred yeast cells include, but are not limited to, the genera Saccharomyces, Pichia, and Kluveromyces. Preferred yeast hosts are S. cerevisiae and P. pastoris. Preferred yeast vectors can contain an origin of replication sequence from a 2T yeast plasmid, an autonomously replication sequence (ARS), a promoter region, sequences for polyadenylation, sequences for transcription termination, and a selectable marker gene. Shuttle vectors for replication in both yeast and E. coli are also included herein. Alternatively, insect cells maybe used as host cells. In a preferred embodiment, the polypeptides of the invention are expressed using a baculovirus expression system (see, Luckow et al., Bio/Technology, 1988, 6, 47, Baculovirus Expression Vectors: A Laboratory Manual, O'Rielly et al. (Eds.), W. H. Freeman and Company, New York, 1992, and U.S. Pat. No. 4,879.236, each of which is incorporated herein by reference in its entirety). In addition, the MAXBAC™ complete baculovirus expression system (Invitrogen) and the BAC-TO-BAC™ System (Life Technologies) can, for example, be used for production in insect cells.

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds that include CDR sequences which specifically recognize a polypeptide of the invention) which bind to MLK7, preferably specific for MLK7, or fragments thereof. Antibody fragments, including Fab, Fab′, F(ab′)2, and Fv, are also provided by the invention. The term “specific for,” when used to describe antibodies of the invention, indicates that the variable regions of the antibodies of the invention recognize and bind MLK7 polypeptides exclusively (i.e., are able to distinguish MLK7 polypeptide from other known MLK polypeptides by virtue of measurable differences in binding affinity, despite the possible existence of localized sequence identity, homology, or similarity between MLK7 and such polypeptides). It will be understood that specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art (see Harlow et al. (Eds.), Antibodies A Laboratory Manual; Cold Spring Harbor Laboratory; Cold Spring Harbor, N.Y. (1988), Chapter 6).

Antibodies of the invention are useful for therapeutic purposes (by modulating activity of MLK7), diagnostic purposes to detect or quantitate MLK7, and purification of MLK7. Kits comprising an antibody of the invention for any of the purposes described herein are also contemplated. In general, a kit of the invention also includes a control antigen for which the antibody is immunospecific. It is contemplated that in other human disease states or abnormal conditions, preventing the expression of, or inhibiting the activity of, MLK7 will be useful in treating disease states or abnormal conditions. It is contemplated that antisense therapy or gene therapy may be applied to regulate the expression or activity of an MLK7 polypeptide.

The present invention is also directed to kits, including pharmaceutical kits. The kits can comprise any of the polynucleotides described above, any of the polypeptides described above, or any antibody which binds to a polypeptide of the invention as described above, as well as a negative control. The kit preferably comprises additional components, such as, for example, instructions, solid support, reagents helpful for quantification, and the like.

Another aspect of the present invention is directed to compositions, including pharmaceutical compositions, comprising any of the polynucleotides or recombinant expression vectors described above and an acceptable carrier or diluent. Preferably, the carrier or diluent is pharmaceutically acceptable. Suitable carriers are described in the most recent edition of Remington's Pharmaceutical Sciences, A. Osol, a standard reference text in this field, which is incorporated herein by reference in its entirety. Preferred examples of such carriers or diluents include, but are not limited to, water, saline, Ringer's solution, dextrose solution, and 5% human serum albumin. Liposomes and non-aqueous vehicles such as fixed oils can also be used. The formulations are sterilized by commonly used techniques.

Specific binding compounds, including natural ligands and synthetic compounds, can be identified or developed using isolated or recombinant MLK7 products, MLK7 variants, or preferably, cells expressing such products. Binding compounds are useful for purifying MLK7 products and detection or quantification of MLK7 products in fluid and tissue samples using known immunological procedures. Binding compounds are also manifestly useful in modulating (i.e., blocking, inhibiting or stimulating) biological activities of MLK7, especially those activities involved in the JNK stress-activated kinase pathway. Thus, compounds that bind MLK7 polynucleotides or polypeptides can be identified.

The polynucleotide and polypeptide sequence information provided by the present invention also makes possible identification of binding compounds with which an MLK7 polypeptide or polynucleotide will interact. Methods to identify binding compounds include solution assays, iii vitro assays wherein an MLK7 polypeptide is immobilized, and cell-based assays. Identification of binding compounds of MLK7 polypeptides provides candidates for therapeutic or prophylactic intervention in pathologies associated with normal and aberrant MLK7 biological activity.

The invention includes several assay systems for identifying MLK7 binding compounds. In solution assays, methods of the invention comprise the steps of i) contacting an MLK7 polypeptide with one or more candidate binding compounds and ii) identifying the compounds that bind to the MLK7 polypeptide. Identification of the compounds that bind the MLK7 polypeptide can be achieved by isolating the MLK7 polypeptide/binding compound complex, and separating the binding compound from the MLK7 polypeptide. An additional step of characterizing the physical, biological, and/or biochemical properties of the binding compound is also contemplated in other embodiments of the invention. In one aspect, the MLK7 polypeptide/binding compound complex can be isolated using an antibody immunospecific for either the MLK7 polypeptide or the candidate binding compound.

In still other embodiments, either the MLK7 polypeptide or the candidate binding compound comprises a label or tag that facilitates its isolation, and methods of the invention to identify binding compounds include a step of isolating the MLK7 polypeptide/binding compound complex through interaction with the label or tag. An exemplary tag of this type is a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention.

In one variation of an in vitro assay, the invention provides a method comprising the steps of i) contacting an immobilized MLK7 polypeptide with a candidate binding compound and ii) detecting binding of the candidate compound to the MLK7 polypeptide. In an alternative embodiment, the candidate binding compound is immobilized and binding of MLK7 is detected. Immobilization can be accomplished using any of the methods well known in the art, including covalent bonding to a support, a bead, or a chromatographic resin, as well as non-covalent, high affinity interactions such as antibody binding, or use of streptavidin/biotin binding wherein the immobilized compound includes a biotin moiety. Detection of binding can be accomplished i) using a radioactive label on the compound that is not immobilized, ii) using of a fluorescent label on the non-immobilized compound, iii) using an antibody immunospecific for the non-immobilized compound, or iv) using a label on the non-immobilized compound that excites a fluorescent support to which the immobilized compound is attached, as well as other techniques well known and routinely practiced in the art.

The invention also provides cell-based assays to identify binding compounds of a MLK7 polypeptide. In one embodiment, the invention provides a method comprising the steps of contacting an MLK7 polypeptide expressed on the surface of a cell with a candidate binding compound and detecting binding of the candidate binding compound to the MLK7 polypeptide. In a preferred embodiment, the detection comprises detecting a calcium flux or other physiological event in the cell caused by the binding of the compound.

Agents that modulate (i.e., increase, decrease, or block) MLK7 activity or expression can be identified by incubating a putative modulator with a cell containing a MLK7 polypeptide or polynucleotide and determining the effect of the putative modulator on MLK7 activity or expression. The selectivity of a compound that modulates the activity of MLK7 can be evaluated by comparing its effects on MLK7 to its effect on other MLK compounds. Selective modulators can include, for example, antibodies and other proteins, peptides, or organic molecules which specifically bind to an MLK7 polypeptide or an MLK7-encoding polynucleotide. Modulators of MLK7 activity will be therapeutically useful in treatment of diseases and physiological conditions in which normal or aberrant MLK7 activity is involved. Compounds identified as a result of the methods described herein (binding compounds and/or modulators of MLK7 polynucleotides and/or polypeptides) can be used in the treatment of diseases and conditions related to the JNK stress-activated kinase pathway, including, but not limited to, neurodegenarative diseases or conditions and inflammatory conditions or diseases. Representative diseases or conditions include, but are not limited to, Alzheimer's disease (Zhu et al., J. Neurochem., 2001, 76,435-441), Huntinton's disease (Yasuda et al., Genes Cells, 1999, 4, 743-756; Liu, J. Biol. Chem., 1998, 273, 28873-28877), multiple sclerosis (Bonetti et al., Am. J. Pathol., 1999, 155, 1433-1438), neuropathic pain (Rausch et al., Neuroscience, 2000, 101, 767-777), Parkinson's disease (Saporito et al., J. Neurochem., 2000, 75, 1200-1208), traumatic brain injury (Raghupathi et al., J. Neurotrauma, 2000, 17, 927-938), rheumatoid arthritis (Schett et al., Arthritis Rheum., 2000,43, 2501-2512), ethanol exposure during neural development (McAlhany et al., Brain Res. Dev., 2000, 119, 209-216), pancreatitis (Hofken et al., Biochem. Biophys. Res. Commun., 2000, 276, 680-685), polycystic kidney disease (Arnould et al., J. Biol. Chem., 1998, 273, 6013-6018), cardiovascular ischaemia (Force et al., Circ. Res., 1996, 78, 947-953), hypertension (Xu et al., J. Clin. Invest., 1996, 97, 508-514), chemotherapy-induced endothelial apoptosis, glioblastoma (Potapova et al., J. Biol. Chem., 2000, 275, 24767-24775), tamoxifen-resistant breast tumors (Schiff et al., J. Natl. Cancer Inst., 2000, 92, 1926-1934), endothelial cell effects resulting from high glucose levels in diabetes mellitus (Ho et al., Circulation, 2000, 101, 2618-2624), and the like. MLK7 polynucleotides and polypeptides, as well as MLK7 modulators, can also be used in diagnostic assays for such diseases or conditions.

Another aspect of the present invention is directed to methods of identifying compounds that bind to either MLK7 or polynucleotides encoding MLK7, comprising contacting MLK7, or a polynucleotide encoding the same, with a compound, and determining whether the compound binds MLK7, or a nucleic acid molecule encoding the same. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include (which may include compounds which are suspected to bind MLK7, or a nucleic acid molecule encoding the same), but are not limited to, extracellular, intracellular, biologic or chemical origin. The MLK7 polypeptide or polynucleotide employed in such a test may either be free in solution, attached to a solid support, borne on a cell surface or located intracellularly or associated with a portion of a cell. One skilled in the art can, for example, measure the formation of complexes between MLK7 and the compound being tested. Alternatively, one skilled in the art can examine the diminution in complex formation between MLK7 and its substrate caused by the compound being tested.

Another aspect of the present invention is directed to methods of identifying compounds that modulate (i.e., increase or decrease) activity of MLK7 comprising contacting MLK7 with a compound, and determining whether the compound modifies activity of MLK7. The activity in the presence of the test compared is measured to the activity in the absence of the test compound. The activity of MLK7 polypeptides of the invention can be determined by, for example, examining the kinase activity or ability to phosphorylate particular substrates including, but not limited to, MKK4 and MKK7.

In preferred embodiments of the invention, methods of screening for compounds that modulate MLK7 activity comprise contacting test compounds with MLK7 and assaying for the presence of a complex between the compound and MLK7. In such assays, the ligand is typically labeled. After suitable incubation, free ligand is separated from that present in bound form, and the amount of free or uncomplexed label is a measure of the ability of the particular compound to bind to MLK7.

Candidate modulators contemplated by the invention include compounds selected from libraries of either potential activators or potential inhibitors. There are a number of different libraries used for the identification of small molecule modulators, including: i) chemical libraries, ii) natural product libraries, and iii) combinatorial libraries comprised of random peptides, oligonucleotides or small organic molecules. Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry. Natural product libraries are collections of microorganisms, animals, plants, or marine organisms which are used to create mixtures for screening by: i) fermentation and extraction of broths from soil, plant or marine microorganisms or ii) extraction of plants or marine organisms. Natural product libraries include polyketides, non-ribosomal peptides, and variants (non-naturally occurring) thereof. Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Other libraries of interest include peptide, protein, peptidomimetic, multiparallel synthetic collection, recombinatorial, and polypeptide libraries. Identification of modulators through use of the various libraries described herein permits modification of the candidate “hit” (or “lead”) to optimize the capacity of the “hit” to modulate activity.

Other assays can be used to identify compounds that bind to or modulate the activity of an MLK7 polypeptide, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 1989, 340, 245-246, and Fields et al., Trends in Genetics, 1994, 10, 286-292, both of which are incorporated herein by reference in their entirety.

In a particular embodiment, the novel compounds identified by the screening methods according to the invention are low molecular weight organic molecules, in which case a composition or pharmaceutical composition can be prepared thereof for oral intake, such as in tablets. The compositions, or pharmaceutical compositions, comprising the nucleic acid molecules, vectors, polypeptides, antibodies and compounds identified by the screening methods described herein, can be prepared for any route of administration including, but not limited to, oral, intravenous, cutaneous, subcutaneous, nasal, intramuscular or intraperitoneal. The nature of the carrier or other ingredients will depend on the specific route of administration and particular embodiment of the invention to be administered. Examples of techniques and protocols that are useful in this context are, inter alia, found in Remington's Pharmaceutical Sciences, 16th edition, Osol, A (ed.), 1980, which is incorporated herein by reference in its entirety.

The dosage of these low molecular weight compounds will depend on the disease state or condition to be treated and other clinical factors such as weight and condition of the human or animal and the route of administration of the compound. For treating human or animals, between approximately 0.5 mg/kg of body weight to 500 mg/kg of body weight, between approximately 1.0 mg/kg of body weight to 100 mg/kg of body weight, or between approximately 10 mg/kg of body weight to 50 mg/kg of body weight of the compound can be administered. Therapy is typically administered at lower dosages and is continued until the desired therapeutic outcome is observed. The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

Another aspect of the present invention is directed to heterodimers containing MLK7 and any of the MLK proteins. There have been reports of leucine zipper-dependent heterodimers between family members. For example, MLK3 can co-immunoprecipitate co-expressed MLK2 (Leung et al., J. Biol. Chem., 1998, 273, 32408-32415). Co-expression in the same cell can result in heterodimers as well as homodimers of these proteins. Heterodimers could have a unique activity compared to the homodimers and represent an additional target for screening in the methods described above.

In order that the invention disclosed herein may be more efficiently understood, examples are provided below. It should be understood that these examples are for illustrative purposes only and are not to be construed as limiting to the invention in any manner. Throughout these examples, molecular cloning reactions, and other standard recombinant DNA techniques, were carried out according to methods described in Maniatis et al., Molecular Cloning—A Laboratory Manual, 2^{nd} ed, Cold Spring Harbor Press (1989) (hereinafter, “Maniatis et al.”), using commercially available enzymes, except where otherwise noted. Examples 1 and 2 below are actual. Example 3 is prophetic.

### EXAMPLES

### Example 1

### Isolation of Polynucleotide Encoding Human MLK7

A. Isolation of cDNA Encoding Human MLK7

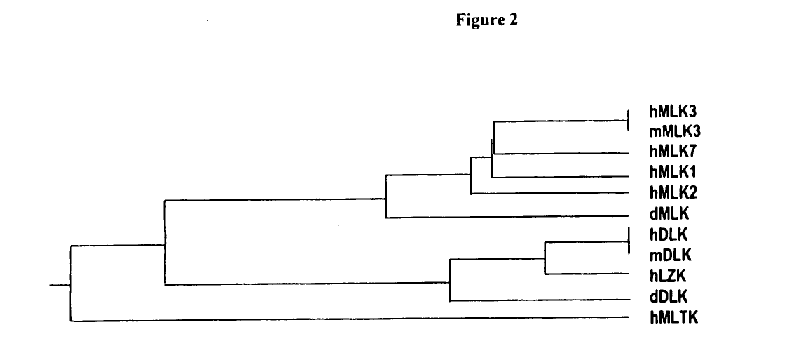

A survey of recently added human genomic DNA sequences revealed a putative seventh member of the mixed lineage kinase family. This human kinase, designated MLK7, was found in Genbank Accession No. AL133380, a completed genomic sequence mapping to q42.2-q43 of chromosome 1. Alignment of the putative kinase domain places this protein in the previously defined MLK1-3 subfamily, with 76% homology to its closest relatives, MLKs 1 and 3. The putative leucine zipper region shown in FIG. 2 would also place this protein in that subfamily, having four putative alpha helical repeats separated by only a 6 amino acid stretch. The full-length protein has the highest homology with MLK1 (44% identity), which was sufficient to allow tentative identification of both the putative N- and C-termini of the protein. Only two recently-deposited MLK7 ESTs were present in Genbank (Accession No. AW813675 from stomach and AW408639 from lymph tissue (germinal center B cells)), requiring additional proof that this genomic region was expressed and not a pseudogene. Clones containing part of the cDNA encoding MLK7 were obtained using PCR screening of pooled human adrenal gland, adult brain, kidney, liver, lymph node, pancreas, prostate, and stomach cDNA libraries (Edge Biosystems). The PCR primers used to screen the libraries were 5′-ATGAAAGAATGCTGGCAACAAGACCCTC-3′ (SEQ ID NO:4) and 5′-AGGTAAACTGATTCGATGTCCATCTTTG-3′ (SEQ ID NO:5). One partial cDNA was isolated repeatedly, encoding a middle portion of the cDNA. Other clones contained genomic sequence or were unrelated. These data are consistent with this cDNA being of very low abundance in these libraries. This sequence was then extended on the 5′ and 3′ ends by PCR based on homology with MLK1. Extension by 5′ RACE was also attempted, but was unsuccessful due to the low abundance and exceptionally high GC content of this cDNA.

The full length human MLK7 cDNA was assembled into the pcDNA6-V5His plasmid (Invitrogen). The plasmid contains the full-length human cDNA (without the stop codon) in frame with a C-terminal epitope and affinity purification fusion partner and is identified as pcDNA6-hMLK7. This plasmid, pcDNA6-hMLK7, was submitted for deposit with American Type Culture Collection, 10801 University Boulevard, Manassas, Va. 20110-2209, on 24 May 2001 under Accession No. ATCC. The full length cDNA and deduced amino acid sequences of human MLK7 are provided in SEQ ID NO:1 and 2, respectively.

B. Structural Comparison to Members of the Mixed Lineage Kinase Family

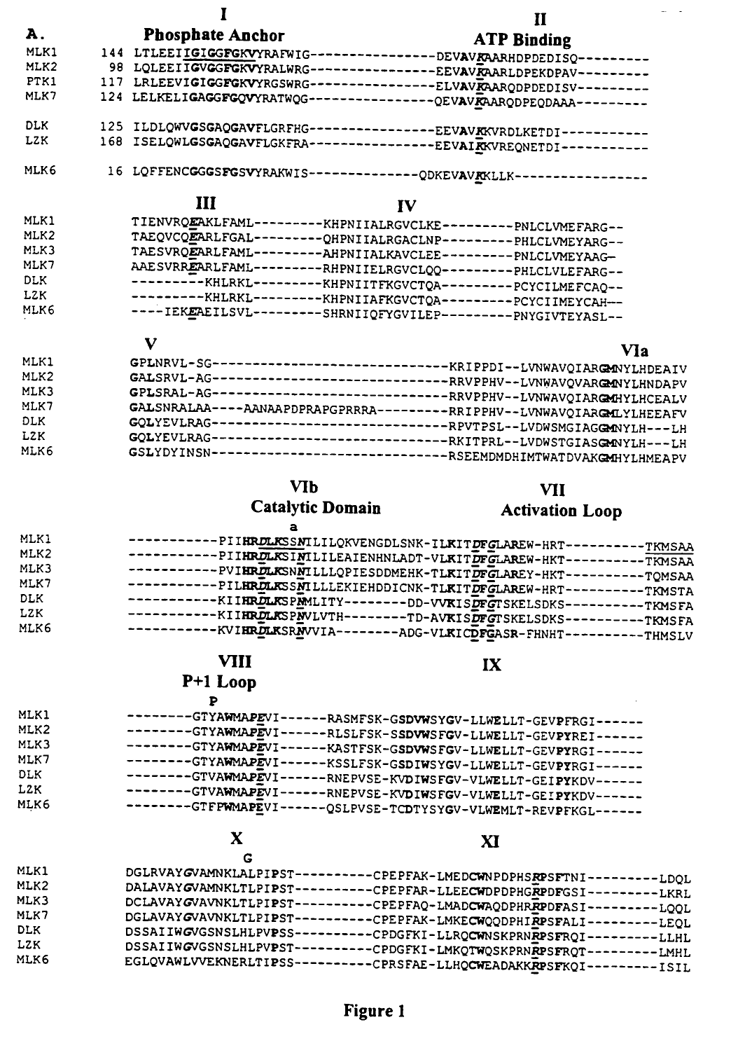

The kinase domains of the six members of the mixed lineage kinase family are shown aligned to the putative kinase domain of MLK7 (FIG. 1). Amino acids that are conserved in tyrosine kinases, serine/threonine kinases or both are shown as designated (consensus amino acids from Hanks et al., Meth. Enzymol., 1991, 200, 38-62). MLK7, like other members of the mixed lineage kinase family, shares homology throughout most of the kinase domain with tyrosine kinases. The presence of lysine in the sequence HRDLKSRN (SEQ ID NO:3) in the catalytic domain of subdomain VIb suggests that, like other members of the MLK family, this would catalyze serine/threonine phosphorylation. Alignment of these kinase domains using the Clustal method (with PAM250 residue weight table) in the MegAlign program (Lasergene DNAstar) predicts MLK7 to be a member of the MLK1-3 subfamily (FIG. 2). The percent homology among the kinase domains of the MLK family is shown in Table 1.

TABLE 1

| Kinase Domain | MLK1 | MLK2 | MLK3 | MLK7 | DLK | LZK | MLK6 |
|---|---|---|---|---|---|---|---|
| MLK1 | — | 75.4% | 77.7% | 75.4% | 42.9% | 41.7% | 42.0% |
| MLK2 | — | — | 76.5% | 73.1% | 44.6% | 42.5% | 41.6% |
| MLK3 | — | — | — | 76.2% | 42.5% | 42.5% | 40.0% |
| MLK7 | — | — | — | — | 38.3% | 39.2% | 40.4% |
| DLK | — | — | — | — | — | 87.1% | 37.1% |
| LZK | — | — | — | — | — | — | 35.4% |
| MLK6 | — | — | — | — | — | — | — |

The full-length MLK7 protein shows weaker homology (using the same analysis as described above) to the family than that of the kinase domain (Table 2).

TABLE 2

| Full Length | MLK1 | MLK2 | MLK3 | MLK7 | DLK | LZK | MLK6α |
|---|---|---|---|---|---|---|---|
| MLK1 | — | 49.0% | 43.6% | 43.1% | 22.2% | 19.4% | 20.1% |
| MLK2 | — | — | 44.3% | 41.8% | 23.6% | 19.7% | 19.6% |
| MLK3 | — | — | — | 44.9% | 22.7% | 20.3% | 18.8% |
| MLK7 | — | — | — | — | 19.1% | 18.6% | 18.5% |
| DLK | — | — | — | — | — | 50.5% | 18.8% |
| LZK | — | — | — | — | — | — | 17.6% |
| MLK6α | — | — | — | — | — | — | — |

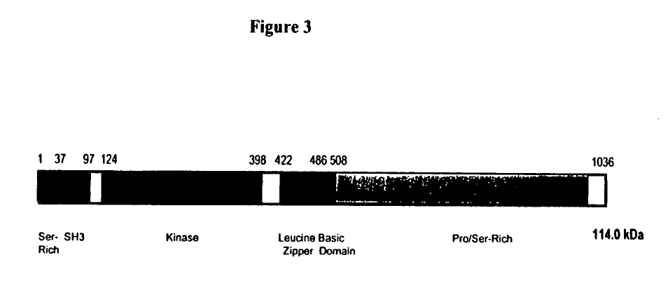

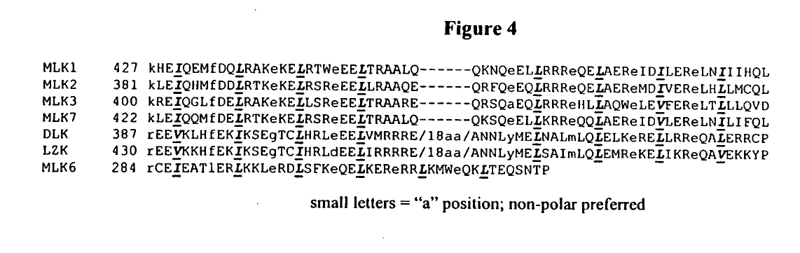

There is a putative SH3 domain N-terminal to the kinase domain. There is also a putative dual leucine zipper region C-terminal to the kinase domain, followed by a small region comprised of a preponderance of basic amino acids similar to the nuclear localization signals from several proteins. The putative leucine zipper region (FIG. 4) also places MLK7 in the MLK1-3 subfamily, having four putative alpha-helical repeats separated by only a six amino acid stretch. The full-length protein has high homology with MLK1 (43.1%), which was sufficient to allow identification of both the putative N- and C-termini of the protein. This homology was used to clone the full-length MLK7 cDNA. The domain structure of MLK7 is shown in FIG. 3. The sequence of the leucine zipper region of MLK7 is shown and compared to other MLK proteins in FIG. 4.

C. Expression Profile of hMLK7

The tissue distribution of hMLK7 was evaluated using the Rapid Scan™ gene expression panel (Origene). Rapid Scan™ is a PCR based approach for using first strand cDNAs derived from 24 different tissues to generate an expression profile in a 96 well plate format. These 24 cDNAs are arrayed in 4-log dilutions enabling amplification and visualization within the linear range of PCR. The protocol requires PCR reactions to be carried out in a 96-well thermal cycler, using gene specific primer pairs and visualizing the amplification on agarose gels. Table 3 shows qualitative data obtained from duplicate screening of the panel.

TABLE 3

| Tissue | Edge Primer Pair |
|---|---|
| brain | ++ |
| heart | + |
| kidney | ++ |
| spleen | − |
| liver | ++ |
| colon | ++ |
| lung | ++ |
| small intestine | + |
| muscle | + |
| stomach | ++ |
| testis | ++ |
| placenta | − |
| salivary gland | ++ |
| thyroid gland | + |
| adrenal gland | ++ |
| pancreas | ++ |
| ovary | + |
| uterus | − |
| prostrate | + |
| skin | − |
| peripheral blood leukocytes | + |
| bone marrow | + |
| fetal brain | + |
| fetal liver | + |

These experiments utilized the primer pair ATGAAAGAATGCTGGCAACAAGACC CTC (SEQ ID NO:4) and AGGTAAACTGATTCGATGTCCATCTTTG (SEQ ID NO:5). A score of ++ in this MLK7 analysis indicated a visible PCR product at the two highest cDNA concentrations. More than half of the tissue screen gave little or no detectable product, which suggested that MLK7 mRNA is not in high abundance in any tissue.

### Example 2

### Recombinant Expression of Biologically Active MLK7

A. JNK Pathway Activation by Full-Length MLK7 Expression in Mammalian Cells

MLK family members overexpressed in mammalian cells activate the cJun-NH2-terminal kinase (JNK) pathway (Hirai et al., 1997, J. Biol. Chem., 272, 15167; Merritt et al., 1999, J. Biol. Chem., 274, 10195). To determine whether full-length MLK7 does the same, CHO cells were co-transfected with plasmids that drive constitutive expression of MLK7 and constitutive expression of JNK1, determining the JNK activity in lysates by ELISA.

The coding region for full-length MLK7 was inserted into the mammalian expression vector pcDNA6V5/HisA (Invitrogen) in-frame with the C-terminal V5 epitope and His₆ affinity purification fusion partner. Each plasmid was co-transfected with a JNK1 expression vector into CHO cells plated in six-well dishes, using the reagent LipofectaminePLUS (Life Technologies) according to the manufacturer's recommendations. Duplicate cultures were transfected for each condition. As a positive control, an expression vector for MLK3 was substituted for MLK7; as a negative control, an empty vector (no protein coding region) was substituted. Two days after transfection, the cells were treated for six hours with CEP11004 at three concentrations (250 nM, 1 μM, and 5 μM), or with DMSO vehicle control, in reduced serum medium (DMEM plus 0.5% fetal bovine serum). At the end of this treatment the cells were washed twice with ice-cold phosphate-buffered saline and lysed with 0.1 ml per well of 1% (v/v) Triton X-100, 20 mM Tris pH 7.6, 50 mM NaCl, protease inhibitor cocktail (1 mM Pefabloc SC, 10 μM E-64, 10 μM leupeptin, 10 μM pepstatin A, 1 mM EDTA), and phosphatase inhibitor cocktail (25 mM β-glycerophosphate, 2 mM activated sodium orthovanadate). The JNK activity in transfected cell lysates was measured using an ELISA-based format as described for receptor tyrosine kinases (Angeles et al., 1996, Anal. Biochem., 236, 49-55). The NH2-terminus of the JNK substrate cJun (residues 1-79) was expressed in E. coli as a GST fusion protein and purified by standard glutathione affinity methods. Smith et al., Gene, 1988, 67, 31-40. A 96-well microtiter plate (FluoroNUNC Maxisorp) was coated with substrate solution (10 μg/ml GST-cJun in Tris-buffered saline), then washed several times with wash buffer (0.05% Tween-20 in Tris-buffered saline). The plate was blocked with block buffer (3% BSA and 0.2% Tween-20 in Tris-buffered saline) for one hour at 37 C, then washed several times with wash buffer. Concentrated assay buffer was then added to each well, followed by lysate corresponding to equal protein for each sample. The kinase reaction was initiated by adding ATP to 50 μM and incubating at 37 C for 15 minutes. The final assay mixture (100 μl per well) contained 20 mM HEPES, pH 7.4, 0.02% BSA, 20 mM MgCl₂, 2 mM DTT, 5 mM EGTA, 25 mM β-glycerophosphate, 0.1 mM activated sodium orthovanadate, and 50 μM ATP. The kinase reaction was terminated by addition of EDTA to 83 mM, and the plate was washed several times with wash buffer. The phosphorylated product was detected by incubation with anti-phospho-cJun (Ser73) antibody (NEB 9164, 1:5000 in block buffer), washing, incubation with goat anti-rabbit IgG-alkaline phosphatase conjugate (Southern Biotechnology Associates #4050-04, 1:2500 dilution in block buffer), washing, and reaction with the fluorogenic alkaline phosphatase substrate 4-methylumbelliferyl phosphate (0.02 mg/ml in 1.0 M diethanolamine, pH 9.6, 5 mM MgCl₂) for 30 minutes at 37 C. The reaction was stopped with 1.0 M dibasic sodium phosphate and the product measured in a microplate reader (Cytofluor, 360 nm excitation, 460 nm emission). A preliminary ELISA was performed to determine the maximum amount of lysate protein that yielded a linear signal. In this experiment, the MLK3 was the limiting sample, maintaining linearity only up to 80 ng of lysate protein. Assays were then performed on 80 ng of each lysate.

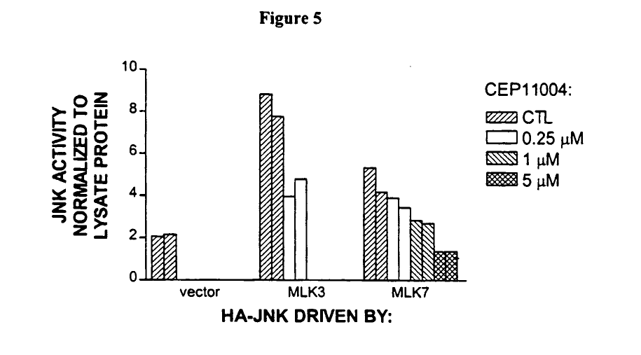

Expression of MLK7 was confirmed by probing Western blots of cell lysates with antibody to the V5 epitope tag (data not shown). MLK7 appeared at an apparent mobility corresponding to 120 kDa (expected 117.4 kDa). Expression of MLK7 elevated JNK activity over the vector control (FIG. 5). Treatment of MLK7-transfected cells with CEP11004 partially reduced JNK activity, just as this treatment does to MLK3-transfected cells. These data confirm that MLK7 activates the JNK pathway in mammalian cells, just like other MLK family members, and just as predicted by the in vitro data.

B. In Vitro Activity of Recombinant GST-MLK7_{KD} and GST-MLK7_{KD/LZ}

A portion of the human MLK7 protein encoding either the kinase domain (amino acids 93-416) or the kinase domain/leucine zipper (amino acids 93-489) were expressed with an NH₂-terminal GST fusion partner in Sf21 insect cells using standard methods (e.g. Meyer et al., J. Neurochem., 1994, 62, 825-833) and purified using glutathione affinity purification methods (Smith et al., Gene, 1988, 67, 31-40). The kinase activities of baculoviral human GST-MLK7_{KD} and GST-MLK7_{KD/LZ} were demonstrated using three different substrates, kinase-dead forms of GST-MKK4 and GST-MKK7, and myelin basic protein (MBP). Additionally, activation of the JNK pathway activation by the two GST-MLK7 forms was shown in vitro. Both GST-MLK7_{KD} and GST-MLK7_{KD/LZ} were able to phosphorylate inactivated MKK4 and, as a consequence of this phosphorylation event, MKK4 was activated and was able to phosphorylate its protein substrate GST-JNK1β1(K55A). Similar results were obtained when inactivated MKK7 was used as the downstream target of MLK7.

C. Phosphorylation of Myelin Basic Protein

The kinase activity of baculoviral GST-MLK7_{KD} and GST-MLK7_{KD/LZ} were assessed separately using the Millipore Multiscreen TCA “in-plate” format as described for protein kinase C (Pitt et al., J. Biomol. Screening, 1996, 1: 47-51). Briefly, each 50-μl assay mixture contained 20 mM Hepes, pH 7.2, 5 mM EGTA, 15 mM MgCl₂, 1 mM DTT,25 mM β-glycerophosphate, 100 μM ATP, 0.25 μCi[y-³²P]ATP, 500 μg/ml myelin basic protein (UBI #13-104), and 0.1-5 μg/ml of baculoviral GST-MLK7_{KD} (or GST-MLK7_{KD/LZ}). Samples were incubated for 15 minutes at 37 C. The reaction was stopped by adding ice cold 50% TCA and the proteins were allowed to precipitate for 30 minutes at 4 C. The plates were then washed with ice cold 25% TCA. Supermix scintillation cocktail was added, and the plates were allowed to equilibrate for 1-2 hours prior to counting using the Wallac MicroBeta 1450 PLUS scintillation counter.

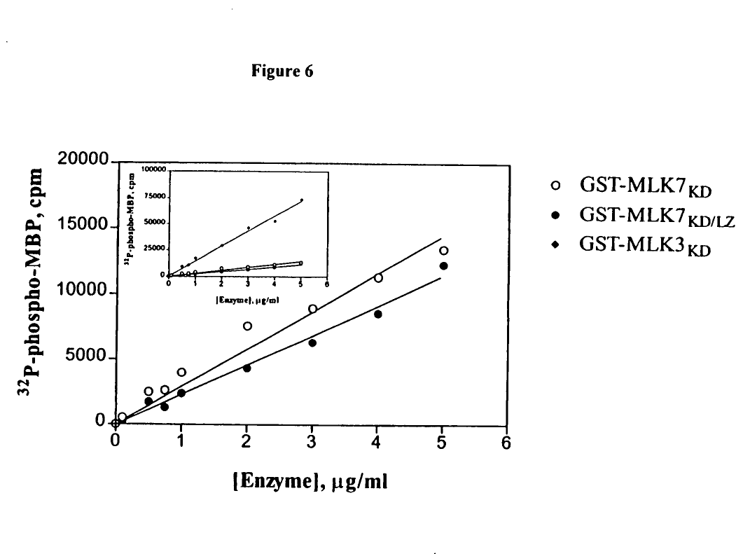

Both GST-MLK7_{KD a}nd GST-MLK7_{KD/LZ} were able to phosphorylate myelin basic protein, a generic protein kinase substrate (FIG. 6). The amount of phosphorylated product formed (reported as radioactive counts) was dependent on the amount of enzyme used in the assay. Baculoviral GST-MLK3_{KD} was used as the control enzyme in this analysis to show phosphorylation of myelin basic protein by a member of the mixed lineage kinase family.

D. Phosphorylation of kinase-inactive GST-MKK4 and GST-MKK7

Kinase-inactive versions of the two known MAPKK proteins in the JNK pathway, MKK4 and MKK7, are expressed as full-length proteins with an N-terminal GST fusion partner in bacteria and purified using standard glutathione affinity purification protocols. Smith et al., Gene, 1988, 67, 31-40. These are used to assess the activity of the MLK7 kinase domain.

Assessment of the kinase activities of baculoviral human GST-MLK7_{KD} and GST-MLK7_{KD/LZ} was carried out using a modification of the ELISA-based assay described for receptor tyrosine kinases (Angeles et al., Anal. Biochem., 1996, 236, 49-55). The 96-well microtiter plate (FluoroNUNC Maxisorp) was coated overnight at 4 C with 20 μg/ml substrate solution (GST-MKK4 (K113A) or GST-MKK7β1(K149A) in Tris-buffered saline). The plate was then blocked with block buffer (3% BSA in Tris-buffered saline containing 0.05% Tween-20) for 1 hour at 37 C. The 100-μl assay mixture containing 20 mM Hepes, pH 7.2, 50 μM ATP, 15 mM MgCl₂, 1 mM DTT, 5 mM EGTA, and 25 mM β-glycerophosphate was then added to the plate. The kinase reaction was initiated by adding 10-100 ng/ml of recombinant human baculoviral GST-MLK7_{KD} or GST-MLK7_{KD/LZ} and the plate was allowed to incubate at 37 C for 15 minutes. The phosphorylated product was detected by adding a phospho-threonine antibody (NEB # 226-1) at a dilution of 1:5000 in block buffer. After a 1-hour incubation at 37 C, 100 μl of europium-N1 labeled anti-rabbit antibody (Wallac # AD0105) at 1:20000 dilution in block buffer was added, and the plate was incubated at 37 C for another hour. A low pH enhancement solution (Wallac # 1244-105) was then added and the plate was gently agitated for 5 minutes at room temperature. The fluorescence of the resulting solution was measured using the Victor 2 Multilabel Counter (Model # 1420-018).

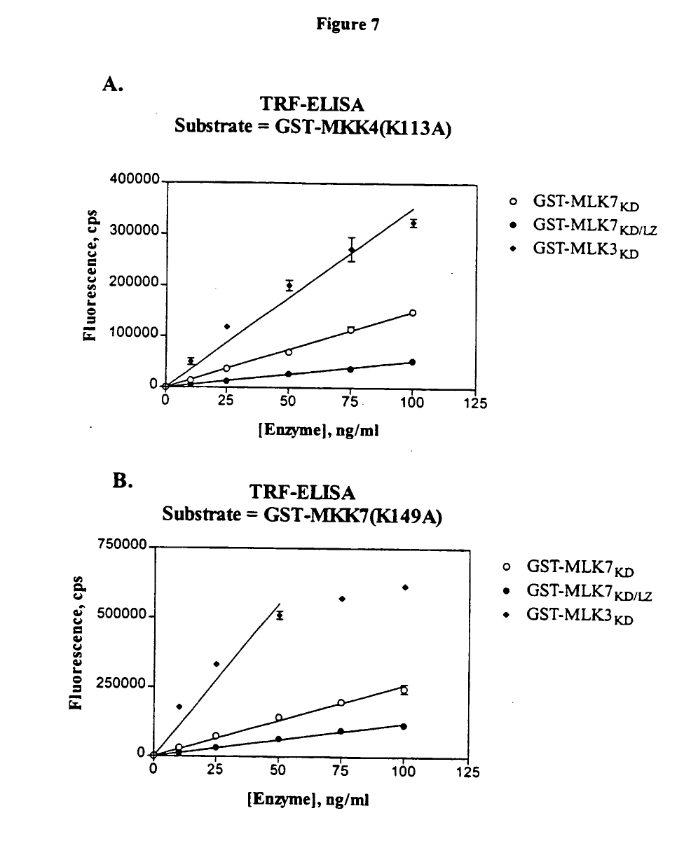

Members of the mixed lineage kinase subfamily of serine/threonine protein kinases have been shown to phosphorylate and activate MKK4 (Rana et al., J. Biol. Chem., 1996, 271, 19025-19028; Hira et al., J. Biol. Chem., 1997, 272, 15167-15173) as well as MKK7 (Hira et al., J. Biol. Chem., 1998, 273, 7406-7412; Merritt et. al., J. Biol. Chem., 1999, 274, 10195-10202). These two MKK proteins were, therefore, utilized to examine the enzymatic activities of baculoviral GST-MLK7_{KD} and GST-MLK7_{KD/LZ}. As shown in FIG. 7, both forms of baculoviral GST-MLK7 catalyzed the phosphorylation of the two MKK protein substrates, GST-MKK4(K113A) and GST-MKK7(K149A). Product formation (reported as fluorescence units) was proportional to the concentration of enzyme used. In terms of specific activity, both GST-MLK7 forms were less active than GST-MLK3KD (control enzyme), with GST-MLK7_{KD} displaying about 2× higher activity than GST-MLK7_{KD/LZ}. Consistent with other MLK reactions, the activities of both GST-MLK7_{KD} and GST-MLK7_{KD/LZ} were inhibited by EDTA, conferring the requirement for metal ions (Mg+2) (data not shown). The ELISA-based assay described here could potentially be used for identifying inhibitors of MLK7.

E. In Vitro Activation of JNK Pathway by GST-MLK7_{KD} and GST-MLK7_{KD/LZ}

Since both forms of MLK7 were able to phosphorylate MKK4 and MKK7 in vitro (FIG. 7), MLK7, like the other members of the MLK family, can activate the JNK/SAPK pathway (Tibbles et al., EMBO J., 1996, 15, 7026-7035; Hira et al., J. Biol. Chem., 1997, 272, 15167-15173; Hira et al., J. Biol. Chem., 1998, 273, 7406-7412). To demonstrate that MLK7 is a direct activator of the MKKs, an in vitro kinase cascade reaction was performed. This system utilized recombinant GST-MLK7, inactivated MKK4/7, and kinase-inactive GST-JNK1β1(K55A) and coupled the MLK7-catalyzed phosphorylation/ activation of MKK4/7 to the phosphorylation of inactive GST-JNK1 by activated MKK4/7.

The assay was performed by first incubating inactivated MKK4/7 with GST-MLK7_{KD} or GST-MLK7_{KD/LZ} in a kinase reaction mixture containing 20 mM Hepes, pH 7.2, 50 μM ATP, 15 mM MgCl₂, 1 mM DTT, 5 mM EGTA, and 25 mM β-glycerophosphate, for 15 minutes at 37 C. GST-MLK3_{KD} was utilized as the control enzyme. An aliquot of this mixture (preactivated MKK4/7; Product 1) was added to a polypropylene, non-treated 96-well microtiter plate containing the MKK4/7 assay mixture. The MKK4/7 reaction utilized the same assay buffer as the GST-MLK7 reaction except for the addition of kinase-dead GST-JNK1β1(K55A). Reaction was allowed to proceed for 15 minutes at 37 C then stopped with 100 mM EDTA (Product 2). Analysis of phosphorylated JNK1 was performed by transferring Product 2 into a FluoroNUNC Maxisorp 96-well microtiter plate. Following a 1 hour incubation at 37 C, the plate was blocked with 3% BSA in TBST. The detection antibody, phospho-specific JNK antibody (Promega # V7931/2), was added and the plate was incubated for an hour at 37 C. Addition of the secondary antibody, Eu—N1 labeled anti-rabbit antibody (Wallac # AD0105) immediately followed, and the plate was incubated at 37 C for another hour. A low pH enhancement solution (Wallac # 1244-105) was then added, and the fluorescence of the resulting solution was measured using the Victor 2 Multilabel Counter (Model # 1420-018).

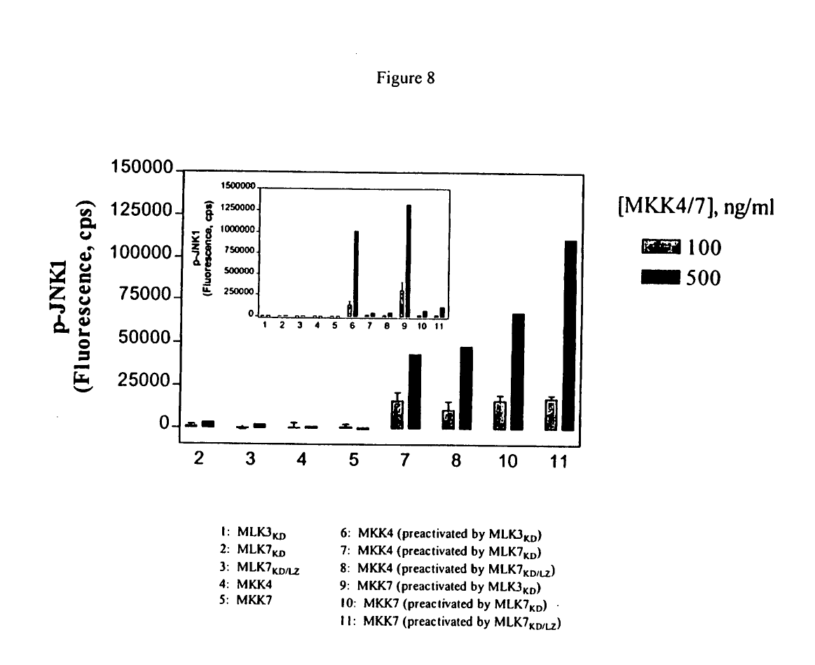

As shown in FIG. 8 (bars 7 and 8), both GST-MLK7_{KD} and GST-MLK7_{KD/LZ} were able to phosphorylate MKK4 and as a consequence of this phosphorylation event, MKK4 was activated and was able to phosphorylate its protein substrate GST-JNK1β1(K55A). Similar results were observed with MKK7 as the downstream target of MLK7 (FIG. 8, bars 10 and 11). Relative to MLK3 (control enzyme; see inset in FIG. 8), the MLK⁷_{KD}- and MLK7_{KD/LZ}-driven JNK activation signals were weaker, reflecting the observed lower specific activities of these enzyme preparations (FIGS. 6 and 7). Overall, the in vitro data indicate that MLK7 can phosphorylate and activate MKK4/7, thereby acting as a MAPKK kinase in the JNK/SAPK pathway.

### Example 3

### Feasibility of using GST-MLK7_{KD} Enzyme Assays to Screen for Inhibitors

The MLK enzymatic activity is further evaluated in the MBP phosphorylation assay and apparent Michaelis constants (Kₘ) for ATP and MBP can be determined. The apparent Kₘ values for ATP and MBP are expected to be about 103 μM and 21 μM, respectively. These values can be compared to values obtained for MLK1-3. Those proteins are also expressed as truncations of either the kinase domain or kinase and leucine zipper domains with N-terminal GST fusion partners.

The initial evaluation of MLK7 activity is performed using the radioactive MBP phosphorylation assay. An ELISA assay is adopted in order to obtain a sufficient signal with lower enzyme concentrations in order to conduct kinetic and inhibition studies.

ELISA Assay Protocol: Assays are performed in 96 well FluoroNunc Maxisorp ELISA plates coated with 10 μg/ml GST-MKK4 (kinase dead mutant) diluted in Tris buffered saline (TBS). Coating is achieved by allowing the MKK4 substrate to stand in the wells for 16 hours at 4 C in a humidified chamber. After coating, excess buffer is aspirated, plates are washed 3 times with TBS containing 0.05% Tween 20 (v/v), and blocked with 3% BSA (w/v) in TBS-T (200 μl/well) for 1 hour at 37 C. All subsequent incubations are carried out using 100 μl/well volumes for one hour at 37 C in a humidified chamber. After blocking, plates are washed 3 times in TBS-T and TBS, respectively. The kinase reaction is performed in 20 mM HEPES, pH 7.4, 30 μM ATP, 15 mM MgCl₂, 1 mM DTT, 0.1 mM Na₃VO₄, 5 mM EGTA, and 25 mM β-glycerophosphate (90 μl/well) and 50 ng/ml GST-MLK7_{KD} (10 μl/well) for 30 minutes at 37 C. As a negative control, 0.5 M EDTA (30 μl/well) is added to the reaction prior to incubation. Plates are washed 3 times in TBS-T and polyclonal antibody 226.1 (New England Biolabs), which detects phosphorylated MKK4, is added to wells at a 1/5000 dilution in blocking buffer. After incubation, plates are washed and alkaline phosphatase conjugated goat anti-rabbit secondary antibody is added at a 1/2500 dilution in blocking buffer. Following a 1-hour incubation and plate washing, 4-methyl umbelliferyl phosphate (0.2 mg/ml final concentration) is added to wells and allowed to develop for 45 minutes. The reaction is terminated with 0.5 M Na₂HPO₄ and read on a fluorescence plate reader at 360 nm excitation wavelength and 460 nm emission wavelength.

Optimization of ELISA: The ELISA is optimized with regard to substrate preference and coating concentration, primary antibody dilution, and development time for the fluorogenic substrate. Magnesium ion requirements, sensitivity to reductants and DMSO can also be evaluated. Both MKK4 and MKK7 kinase dead mutants are suitable substrates for phosphorylation by GST-MLK7_{KD} and are expected to demonstrate a dose-dependent increase in signal strength with increasing substrate concentration. Subsequent experiments are routinely performed using 20 μg/ml MKK4 as the substrate. Primary antibody 226.1 (New England BioLabs) dilution is assayed, and under conditions employed for the ELISA, a 1/5000 dilution was determined to be optimal.

Two reference kinase inhibitors, K252a and staurosporine, can be examined for their ability to inhibit GST-MLK7_{KD} activity.

The disclosures of each patent, patent application and publication cited or described in this document are hereby incorporated herein by reference, in their entirety.

Various modification of the invention, in addition to those described herein, will be apparent to those skilled in the art from the foregoing description. Such modifications are also intended to fall within the scope of the appended claims.

| | |
|---|---|
| Polynucleotide sequence (SEQ ID NO:1) | |
| ATG GCT TTG CGG GGC GCC GCG GGA GCG ACC GAC ACC CCG GTG | 42 |
| Met Ala Leu Arg Gly Ala Ala Gly Ala Thr Asp Thr Pro Val | |
| TCC TCG GCC GGG GGA GCC CCC GGC GGC TCA GCG TCC TCG TCG | 84 |
| Ser Ser Ala Gly Gly Ala Pro Gly Gly Ser Ala Ser Ser Ser | |
| TCC ACC TCC TCG GGC GGC TCG GCC TCG GCG GGC GCG GGG CTG | 126 |
| Ser Thr Ser Ser Gly Gly Ser Ala Ser Ala Gly Ala Gly Leu | |
| TGG GCC GCG CTC TAT GAC TAC GAG GCT CGC GGC GAG GAC GAG | 168 |
| Trp Ala Ala Leu Tyr Asp Tyr Glu Ala Arg Gly Glu Asp Glu | |
| CTG AGC CTG CGG CGC GGC CAG CTG GTG GAG GTG CTG TCG CAG | 210 |
| Leu Ser Leu Arg Arg Gly GLn Leu Val Glu Val Leu Ser Gln | |
| GAC GCC GCC GTG TCG GGC GAC GAG GGC TGG TGG GCA GGC CAG | 252 |
| Asp Ala Ala Val Ser Gly Asp Glu Gly Trp Trp Ala Gly Gln | |
| GTG CAG CGG CGC CTC GGC ATC TTC CCC GCC AAC TAC GTG GCT | 294 |
| Val Gln Arg Arg Leu Gly Ile Phe Pro Ala Asn Tyr Val Ala | |
| CCC TGC CGC CCG GCC GCC AGC CCC GCG CCG CCG CCC TCG CGG | 336 |
| Pro Cys Arg Pro Ala Ala Ser Pro Ala Pro Pro Pro Ser Arg | |
| CCC AGC TCC CCG GTA CAC GTC GCC TTC GAG CGG CTG GAG CTG | 378 |
| Pro Ser Ser Pro Val His Val Ala Phe Glu Arg Leu Glu Leu | |
| AAG GAG CTC ATC GGC GCT GGG GGC TTC GGG CAG GTG TAC CGC | 420 |
| Lys Glu Leu Ile Gly Ala Gly Gly Phe Gly Gln Val Tyr Arg | |
| GCC ACC TGG CAG GGC CAG GAG GTC GCC GTG AAG GCG GCG CGC | 462 |
| Ala Thr Trp Gln Gly Gln Glu Val Ala Val Lys Ala Ala Arg | |
| CAG GAC CCG GAG CAG GAC GCG GCG GCG GCT GCC GAG AGC GTG | 504 |
| Gln Asp Pro Glu Gln Asp Ala Ala Ala Ala Ala Glu Ser Val | |
| CGG CGC GAG GCT CGG CTC TTC GCC ATG CTG CGG CAC CCC AAC | 546 |
| Arg Arg Glu Ala Arg Leu Phe Ala Met Leu Arg His Pro Asn | |
| ATC ATC GAG CTG CGC GGC GTG TGC CTG CAG CAG CCG CAC CTC | 588 |
| Ile Ile Glu Leu Arg Gly Val Cys Leu Gln Gln Pro His Leu | |
| TGC CTG GTG CTG GAG TTC GCC CGC GGC GGA GCG CTC AAC CGA | 630 |
| Cys Leu Val Leu Glu Phe Ala Arg Gly Gly Ala Leu Asn Arg | |
| GCG CTG GCC GCT GCC AAC GCC GCC CCG GAC CCG CGC GCG CCC | 672 |
| Ala Leu Ala Ala Ala Asn Ala Ala Pro Asp Pro Arg Ala Pro | |
| GCC CCC CGC CGC GCG CGC CGC ATC CCT CCG CAC GTG CTG GTC | 714 |
| Gly Pro Arg Arg Ala Arg Arg Ile Pro Pro His Val Leu Val | |
| AAC TGG GCC GTG CAG ATA GCG CGG GGC ATG CTC TAC CTG CAT | 756 |
| Asn Trp Ala Val Gln Ile Ala Arg Gly Met Leu Tyr Leu His | |
| GAG GAG GCC TTC GTG CCC ATC CTG CAC CGG GAC CTC AAG TCC | 798 |
| Glu Glu Ala Phe Val Pro Ile Leu His Arg Asp Leu Lys Ser | |
| AGC AAC ATT TTG CTA CTT GAG AAG ATA GAA CAT GAT GAC ATC | 840 |
| Ser Asn Ile Leu Leu Leu Glu Lys Ile Glu His Asp Asp Ile | |
| TGC AAT AAA ACT TTG AAG ATT ACA GAT TTT GGG TTG GCG AGG | 882 |
| Cys Asn Lys Thr Leu Lys Ile Thr Asp Phe Gly Leu Ala Arg | |
| GAA TGG CAC AGG ACC ACC AAA ATG AGC ACA GCA GGC ACC TAT | 924 |
| Glu Trp His Arg Thr Thr Lys Met Ser Thr Ala Gly Thr Tyr | |
| GCC TGG ATG GCC CCC GAA GTG ATC AAG TCT TCC TTG TTT TCT | 966 |
| Ala Trp Met Ala Pro Glu Val Ile Lys Ser Ser Leu Phe Ser | |
| AAG GGA AGC GAC ATC TGG AGC TAT GGA GTG CTG CTG TGG GAA | 1008 |
| Lys Gly Ser Asp Ile Trp Ser Tyr Gly Val Leu Leu Trp Glu | |
| CTG CTC ACC GGA GAA GTC CCC TAT CGG GGC ATT GAT GGC CTC | 1050 |
| Leu Leu Thr Gly Glu Val Pro Tyr Arg Gly Ile Asp Gly Leu | |
| GCC GTG GCT TAT GGG GTA GCA GTC AAT AAA CTC ACT TTG CCC | 1092 |
| Ala Val Ala Tyr Gly Val Ala Val Asn Lys Leu Thr Leu Pro | |
| ATT CCA TCC ACC TGC CCT GAG CCG TTT GCC AAG CTC ATG AAA | 1134 |
| Ile Pro Ser Thr Cys Pro Glu Pro Phe Ala Lys Leu Met Lys | |
| GAA TGC TGG CAA CAA GAC CCT CAT ATT CGT CCA TCG TTT GCC | 1176 |
| Glu Cys Trp Gln Gln Asp Pro His Ile Arg Pro Ser Phe Ala | |
| TTA ATT CTC GAA CAG TTG ACT GCT ATT GAA GGG GCA GTG ATG | 1218 |
| Leu Ile Leu Glu Gln Leu Thr Ala Ile Glu Gly Ala Val Met | |
| ACT GAG ATG CCT CAA GAA TCT TTT CAT TCC ATG CAA GAT GAC | 1260 |
| Thr Glu Met Pro Gln Glu Ser Phe His Ser Met Gln Asp Asp | |
| TGG AAA CTA GAA ATT CAA CAA ATG TTT GAT GAG TTG AGA ACA | 1302 |
| Trp Lys Leu Glu Ile Gln Gln Met Phe Asp Glu Leu Arg Thr | |
| AAG GAA AAG GAG CTG CGA TCC CGG GAA GAG GAG CTG ACT CGG | 1344 |
| Lys Glu Lys Glu Leu Arg Ser Arg Glu Glu Glu Leu Thr Arg | |
| GCG GCT CTG CAG CAG AAG TCT CAG GAG GAG CTG CTA AAG CGG | 1386 |
| Ala Ala Leu Gln Gln Lys Ser Gln Glu Glu Leu Leu Lys Arg | |
| CGT GAG CAG CAG CTG GCA GAG CGC GAG ATC GAC GTG CTG GAG | 1428 |
| Arg Glu Gln Gln Leu Ala Glu Arg Glu Ile Asp Val Leu Glu | |
| CGG GAA CTT AAC ATT CTG ATA TTC CAG CTA AAC CAG GAG AAG | 1470 |
| Arg Glu Leu Asn Ile Leu Ile Phe Gln Leu Asn Gln Glu Lys | |
| CCC AAG GTA AAG AAG AGG AAG GGC AAG TTT AAG AGA AGT CGT | 1512 |
| Pro Lys Val Lys Lys Arg Lys Gly Lys Phe Lys Arg Ser Arg | |
| TTA AAG CTC AAA GAT GGA CAT CGA ATC AGT TTA CCT TCA GAT | 1554 |
| Leu Lys Leu Lys Asp Gly His Arg Ile Ser Leu Pro Ser Asp | |
| TTC CAG CAC AAG ATA ACC GTG CAG GCC TCT CCC AAC TTG GAC | 1596 |
| Phe Gln His Lys Ile Thr Val Gln Ala Ser Pro Asn Leu Asp | |
| AAA CGG CGG AGC CTG AAC AGC AGC AGT TCC AGT CCC CCG AGC | 1638 |
| Lys Arg Arg Ser Leu Asn Ser Ser Ser Ser Ser Pro Pro Ser | |
| AGC CCC ACA ATG ATG CCC CGA CTC CGA GCC ATA CAG TTG ACT | 1680 |
| Ser Pro Thr Met Met Pro Arg Leu Arg Ala Ile Gln Leu Thr | |
| TCA GAT GAA AGC AAT AAA ACT TGG GGA AGG AAC ACA GTC TTT | 1722 |
| Ser Asp Glu Ser Asn Lys Thr Trp Gly Arg Asn Thr Val Phe | |
| CGA CAA GAA GAA TTT GAG GAT GTA AAA AGG AAT TTT AAG AAA | 1764 |
| Arg Gln Glu Glu Phe Glu Asp Val Lys Arg Asn Phe Lys Lys | |
| AAA GGT TGT ACC TGG GGA CCA AAT TCC ATT CAA ATG AAA GAT | 1806 |
| Lys Gly Cys Thr Trp Gly Pro Asn Ser Ile Gln Met Lys Asp | |
| AGA ACA GAT TGC AAA GAA AGG ATA AGA CCT CTC TCC GAT GGC | 1848 |
| Arg Thr Asp Cys Lys Glu Arg Ile Arg Pro Leu Ser Asp Gly | |
| AAC AGT CCT TGG TCA ACT ATC TTA ATA AAA AAT CAG AAA ACC | 1890 |
| Asn Ser Pro Trp Ser Thr Ile Leu Ile Lys Asn Gln Lys Thr | |
| ATG CCC TTG GCT TCA TTG TTT GTG GAC CAG CCA GGG TCC TGT | 1932 |
| Met Pro Leu Ala Ser Leu Phe Val Asp Gln Pro Gly Ser Cys | |
| GAA GAG CCA AAA CTT TCC CCT GAT GGA TTA GAA CAC AGA AAA | 1974 |
| Glu Glu Pro Lys Leu Ser Pro Asp Gly Leu Glu His Arg Lys | |
| CCA AAA CAA ATA AAA TTG CCT AGT CAG GCC TAC ATT GAT CTA | 2016 |
| Pro Lys Gln Ile Lys Leu Pro Ser Gln Ala Tyr Ile Asp Leu | |
| CCT CTT GGG AAA GAT GCT CAG ACA GAG AAT CCT GCA GAA GCT | 2058 |
| Pro Leu Gly Lys Asp Ala Gln Arg Glu Asn Pro Ala Glu Ala | |
| GAA AGC TGG GAG GAG GCA GCC TCT GCG AAT GCT GCC ACA GTC | 2100 |
| Glu Ser Trp Glu Glu Ala Ala Ser Ala Asn Ala Ala Thr Val | |
| TCC ATT GAG ATG ACT CCT ACG AAT AGT CTG AGT AGA TCC CCC | 2142 |
| Ser Ile Glu Mer Thr Pro Thr Asn Ser Leu Ser Arg Ser Pro | |
| CAG AGA AAG AAA ACG GAG TCA GCT CTG TAT GGG TGC ACC GTC | 2184 |
| Gln Arg Lys Lys Thr Glu Ser Ala Leu Tyr Gly Cys Thr Val | |
| CTT CTG GCA TCG GTG GCT CTG GGA CTG GAC CTC AGA GAG CTT | 2226 |
| Leu Leu Ala Ser Val Ala Leu Gly Leu Asp Leu Arg Glu Leu | |
| CAT AAA GCA CAG GCT GCT GAA GAA CCG TTG CCC AAG GAA GAG | 2268 |
| His Lys Ala Gln Ala Ala Glu Glu Pro Leu Pro Lys Glu Glu | |
| AAG AAG AAA CGA GAG GGA ATC TTC CAG CGG GCT TCC AAG TCC | 2310 |
| Lys Lys Lys Arg Glu Gly Ile Phe Gln Arg Ala Ser Lys Ser | |
| CGC AGA AGC GCC AGT CCT CCC ACA AGC CTG CCA TCC ACC TGT | 2352 |
| Arg Arg Ser Ala Ser Pro Pro Thr Ser Leu Pro Ser Thr Cys | |
| GGG GAG GCC AGC AGC CCA CCC TCC CTG CCA CTG TCA AGT GCC | 2394 |
| Gly Glu Ala Ser Ser Pro Pro Ser Leu Pro Leu Ser Ser Ala | |
| CTG GGC ATC CTC TCC ACA CCT TCT TTC TCC ACA AAG TGC CTG | 2436 |
| Leu Gly Ile Leu Ser Thr Pro Ser Phe Ser Thr Lys Cys Leu | |
| CTG CAG ATG GAC AGT GAA GAT CCA CTG GTG GAC AGT GCA CCT | 2478 |
| Leu Gln Met Asp Ser Glu Asp Pro Leu Val Asp Ser Ala Pro | |
| GTC ACT TGT GAC TCT GAG ATG CTC ACT CCG GAT TTT TGT CCC | 2520 |
| Val Thr Cys Asp Ser Glu Met Leu Thr Pro Asp Phe Cys Pro | |
| ACT GCC CCA GGA AGT GGT CGT GAG CCA GCC CTC ATG CCA AGA | 2562 |
| Thr Ala Pro Gly Ser Gly Arg Glu Pro Ala Leu Met Pro Arg | |
| CTT GAC ACT GAT TGT AGT GTA TCA AGA AAC TTG CCG TCT TCC | 2604 |
| Leu Asp Thr Asp Cys Ser Val Ser Arg Asn Leu Pro Ser Ser | |
| TTC CTA CAG CAG ACA TGT GGG AAT GTA CCT TAC TGT GCT TCT | 2646 |
| Phe Leu Gln Gln Thr Cys Gly Asn Val Pro Tyr Cys Ala Ser | |
| TCA AAA CAT AGA CCG TCA CAT CAC AGA CGG ACC ATG TCT GAT | 2688 |
| Ser Lys His Arg Pro Ser His His Arg Arg Thr Met Ser Asp | |
| GGA AAT CCG ACC CCA ACT GGT CCA ACT ATT ATC TCA GCC ACT | 2730 |
| Gly Asn Pro Thr Pro Thr Gly Ala Thr Ile Ile Ser Ala Thr | |
| GGA GCC TCT GCA CTG CCA CTC TGC CCC TCA CCT GCT CCT CAC | 2772 |
| Gly Ala Ser Ala Leu Pro Leu Cys Pro Ser Pro Ala Pro His | |
| AGT CAT CTG CCA AGG GAG GTC TCA CCC AAG AAG CAC AGC ACT | 2814 |
| Ser His Leu Pro Arg Glu Val Ser Pro Lys Lys His Ser Thr | |
| GTC CAC ATC GTG CCT CAG CGT CGC CCT GCC TCC CTG AGA AGC | 2856 |
| Val His Ile Val Pro Gln Arg Arg Pro Ala Ser Leu Arg Ser | |
| CGC TCA GAT CTG CCT CAG GCT TAC CCA CAG ACA GCA GTG TCT | 2898 |
| Arg Ser Asp Leu Pro Gln Ala Tyr Pro Gln Thr Ala Val Ser | |
| CAG CTG GCA CAG ACT GCC TGT GTA GTG GGT CGC CCA GGA CCA | 2940 |
| Gln Leu Ala Gln Thr Ala Cys Val Val Gly Arg Pro Gly Pro | |
| CAT CCC ACC CAA TTC CTC GCT GCC AAG GAG AGA ACT AAA TCC | 2982 |
| His Pro Thr Gln Phe Leu Ala Ala Lys Glu Arg Thr Lys Ser | |
| CAT GTG CCT TCA TTA CTG GAT GCT GAC GTG GAA GGT CAG ACG | 3024 |
| His Val Pro Ser Leu Leu Asp Ala Asp Val Glu Gly Gln Ser | |
| AGG GAC TAC ACT GTG CCA CTG TGC AGA ATG AGG AGC AAA ACC | 3066 |
| Arg Asp Tyr Thr Val Pro Leu Cys Arg Met Arg Ser Lys Thr | |
| AGC CGG CCA TCT ATA TAT GAA CTG GAG AAA GAA TTC CTC TCT | 3108 |
| Ser Arg Pro Ser Ile Tyr Glu Leu Glu Lys Glu Phe Leu Ser | |
| TAA | 3111 |
| Stop | |
| Polypeptide sequence (SEQ ID NO:2) | |
| Met Ala Leu Arg Gly Ala Ala Gly Ala Thr Asp Thr Pro Val | |
| Ser Ser Ala Gly Gly Ala Pro Gly Gly Ser Ala Ser Ser Ser | |
| Ser Thr Ser Ser Gly Gly Ser Ala Ser Ala Gly Ala Gly Leu | |
| Trp Ala Ala Leu Tyr Asp Tyr Glu Ala Arg Gly Glu Asp Glu | |
| Leu Ser Leu Arg Arg Gly Gln Leu Val Glu Val Leu Ser Gln | |
| Asp Ala Ala Val Ser Gly Asp Glu Gly Trp Trp Ala Gly Gln | |
| Val Gln Arg Arg Leu Gly Ile Phe Pro Ala Asn Tyr Val Ala | |
| Pro Cys Arg Pro Ala Ala Ser Pro Ala Pro Pro Pro Ser Arg | |
| Pro Ser Ser Pro Val His Val Ala Phe Glu Arg Leu Glu Leu | |
| Lys Glu Leu Ile Gly Ala Gly Gly Phe Gly Gln Val Tyr Arg | |
| Ala Thr Trp Gln Gly Gln Glu Val Ala Val Lys Ala Ala Arg | |
| Gln Asp Pro Glu Gln Asp Ala Ala Ala Ala Ala Glu Ser Val | |
| Arg Arg Glu Ala Arg Leu Phe Ala Met Leu Arg His Pro Asn | |
| Ile Ile Glu Leu Arg Gly Val Cys Leu Gln Gln Pro His Leu | |
| Cys Leu Val Leu Glu Phe Ala Arg Gly Gly Ala Leu Asn Arg | |
| Ala Leu Ala Ala Ala Asn Ala Ala Pro Asp Pro Arg Ala Pro | |
| Gly Pro Arg Arg Ala Arg Arg Ile Pro Pro His Val Leu Val | |
| Asn Trp Ala Val Gln Ile Ala Arg Gly Met Leu Tyr Leu His | |
| Glu Glu Ala Phe Val Pro Ile Leu His Arg Asp Leu Lys Ser | |
| Ser Asn Ile Leu Leu Leu Glu Lys Ile Glu His Asp Asp Ile | |
| Cys Asn Lys Thr Leu Lys Ile Thr Asp Phe Gly Leu Ala Arg | |
| Glu Trp His Arg Thr Thr Lys Met Ser Thr Ala Gly Thr Tyr | |
| Ala Trp Met Ala Pro Glu Val Ile Lys Ser Ser Leu Phe Ser | |
| Lys Gly Ser Asp Ile Trp Ser Tyr Gly Val Leu Leu Trp Glu | |
| Leu Leu Thr Gly Glu Val Pro Tyr Arg Gly Ile Asp Gly Leu | |
| Ala Val Ala Tyr Gly Val Ala Val Asn Lys Leu Thr Leu Pro | |
| Ile Pro Ser Thr Cys Pro Glu Pro Phe Ala Lys Leu Met Lys | |
| Glu Cys Trp Gln Gln Asp Pro His Ile Arg Pro Ser Phe Ala | |
| Leu Ile Leu Glu Gln Leu Thr Ala Ile Glu Gly Ala Val Met | |
| Thr Glu Met Pro Gln Glu Ser Phe His Ser Met Gln Asp Asp | |
| Trp Lys Leu Glu Ile Gln Gln Met Phe Asp Glu Leu Arg Thr | |
| Lys Glu Lys Glu Leu Arg Ser Arg Glu Glu Glu Leu Thr Arg | |
| Ala Ala Leu Gln Gln Lys Ser Gln Glu Glu Leu Leu Lys Arg | |
| Arg Glu Gln Gln Leu Ala Glu Arg Glu Ile Asp Val Leu Glu | |
| Arg Glu Leu Asn Ile Leu Ile Phe Gln Leu Asn Gln Glu Lys | |
| Pro Lys Val Lys Lys Arg Lys Gly Lys Phe Lys Arg Ser Arg | |
| Leu Lys Leu Lys Asp Gly His Arg Ile Ser Leu Pro Ser Asp | |
| Phe Gln His Lys Ile Thr Val Gln Ala Ser Pro Asn Leu Asp | |
| Lys Arg Arg Ser Leu Asn Ser Ser Ser Ser Ser Pro Pro Ser | |
| Ser Pro Thr Met Met Pro Arg Leu Arg Ala Ile Gln Leu Thr | |
| Ser Asp Glu Ser Asn Lys Thr Trp Gly Arg Asn Thr Val Phe | |
| Arg Gln Glu Glu Phe Glu Asp Val Lys Arg Asn Phe Lys Lys | |
| Lys Gly Cys Thr Trp Gly Pro Asn Ser Ile Gln Met Lys Asp | |
| Arg Thr Asp Cys Lys Glu Arg Ile Arg Pro Leu Ser Asp Gly | |
| Asn Ser Pro Trp Ser Thr Ile Leu Ile Lys Asn Gln Lys Thr | |
| Met Pro Leu Ala Ser Leu Phe Val Asp Gln Pro Gly Ser Cys | |
| Glu Glu Pro Lys Leu Ser Pro Asp Gly Leu Glu His Arg Lys | |
| Pro Lys Gln Ile Lys Leu Pro Ser Gln Ala Tyr Ile Asp Leu | |
| Pro Leu Gly Lys Asp Ala Gln Arg Glu Asn Pro Ala Glu Ala | |
| Glu Ser Trp Glu Glu Ala Ala Ser Ala Asn Ala Ala Thr Val | |
| Ser Ile Glu Mer Thr Pro Thr Asn Ser Leu Ser Arg Ser Pro | |
| Gln Arg Lys Lys Thr Glu Ser Ala Leu Tyr Gly Cys Thr Val | |
| Leu Leu Ala Ser Val Ala Leu Gly Leu Asp Leu Arg Glu Leu | |
| His Lys Ala Gln Ala Ala Glu Glu Pro Leu Pro Lys Glu Glu | |
| Lys Lys Lys Arg Glu Gly Ile Phe Gln Arg Ala Ser Lys Ser | |
| Arg Arg Ser Ala Ser Pro Pro Thr Ser Leu Pro Ser Thr Cys | |
| Gly Glu Ala Ser Ser Pro Pro Ser Leu Pro Leu Ser Ser Ala | |
| Leu Gly Ile Leu Ser Thr Pro Ser Phe Ser Thr Lys Cys Leu | |
| Leu Gln Met Asp Ser Glu Asp Pro Leu Val Asp Ser Ala Pro | |
| Val Thr Cys Asp Ser Glu Met Leu Thr Pro Asp Phe Cys Pro | |
| Thr Ala Pro Gly Ser Gly Arg Glu Pro Ala Leu Met Pro Arg | |
| Leu Asp Thr Asp Cys Ser Val Ser Arg Asn Leu Pro Ser Ser | |
| Phe Leu Gln Gln Thr Cys Gly Asn Val Pro Tyr Cys Ala Ser | |
| Ser Lys His Arg Pro Ser His His Arg Arg Thr Met Ser Asp | |
| Gly Asn Pro Thr Pro Thr Gly Ala Thr Ile Ile Ser Ala Thr | |
| Gly Ala Ser Ala Leu Pro Leu Cys Pro Ser Pro Ala Pro His | |
| Ser His Leu ProArg Glu Val Ser Pro Lys Lys His Ser Thr | |
| Val His Ile Val Pro Gln Arg Arg Pro Ala Ser Leu Arg Ser | |
| Arg Ser Asp Leu Pro Gln Ala Tyr Pro Gln Thr Ala Val Ser | |
| Gln Leu Ala Gln Thr Ala Cys Val Val Gly Arg Pro Gly Pro | |
| His Pro Thr Gln Phe Leu Ala Ala Lys Glu Arg Thr Lys Ser | |
| His Val Pro Ser Leu Leu Asp Ala Asp Val Glu Gly Gln Ser | |
| Arg Asp Tyr Thr Val Pro Leu Cys Arg Met Arg Ser Lys Thr | |
| Ser Arg Pro Ser Ile Tyr Glu Leu Glu Lys Glu Phe Leu Ser | |

1. An isolated polynucleotide comprising SEQ ID NO:1.

2. An isolated polynucleotide comprising a sequence that encodes a polypeptide comprising SEQ ID NO:2.

3. (canceled)

4. An expression vector comprising the polynucleotide of claim 1.

5. The expression vector of claim 4 wherein said vector is a plasmid or viral particle.

6. A host cell transformed with a vector of claim 4.

7. The transformed host cell of claim 6 wherein said cell is a bacterial cell, yeast cell, insect cell, or mammalian cell.

8. A method of producing a polypeptide comprising SEQ ID NO:2 comprising the steps of:
a) introducing a recombinant expression vector of claim 4 into a compatible host cell;
b) growing said host cell under conditions for expression of said polypeptide; and
c) recovering said polypeptide.

9. A composition comprising a polynucleotide of claim 1 and a carrier or diluent.

10. A composition comprising a recombinant expression vector of claim 4 and a carrier or diluent.

11. An isolated polypeptide encoded by a polynucleotide of claim 1.

12. An isolated polypeptide comprising SEQ ID NO:2.

13. The polypeptide of claim 11 wherein said polypeptide comprises SEQ ID NO:2.

14. A composition comprising a polypeptide of claim 11 and a carrier or diluent.

15. A composition comprising a polypeptide of claim 12 and a carrier or diluent.

16. An isolated antibody that binds to an epitope on a polypeptide of claim 12.

17. The antibody of claim 16 wherein said antibody is a monoclonal antibody.

18. A composition comprising an antibody of claim 16 and a carrier or diluent.

19. A kit comprising an antibody that binds to a polypeptide of claim 12.

20. A kit comprising a polynucleotide of claim 1.

21. A kit comprising a polypeptide of claim 12.

22. A method of identifying a compound that binds a polypeptide of claim 12 comprising the steps of:
a) contacting said polypeptide with a compound; and
b) determining whether said compound binds to said polypeptide.

23. The method of claim 22 wherein binding of said compound to said polypeptide is determined by a protein binding assay.

24. The method of claim 23 wherein said protein binding assay is selected from the group consisting of a gel-shift assay, Western blot, radiolabeled competition assay, phage-based expression cloning, co-fractionation by chromatography, co-precipitation, cross linking, interaction trap/two hybrid analysis, southwestern analysis, and ELISA.

25. A method of identifying a compound that binds a polynucleotide of claim 1 comprising the steps of:
a) contacting said polynucleotide with a compound; and
b) determining whether said compound binds said polynucleotide.

26. The method of claim 24 wherein binding is determined by a gel-shift assay.

27. A method for identifying a compound that modulates the activity of a polypeptide of claim 12 comprising the steps of:
a) contacting said polypeptide with a compound; and
b) determining whether said polypeptide activity has been modulated.

28. The method of claim 27 wherein said activity is phosphorylation of a substrate.

29. The method of claim 28 wherein said activity is JNK activation.

30. A compound identified by the method of claim 22.

31. A compound identified by the method of claim 25.

32. A compound identified by the method of claim 27.


SEQUENCE LISTING (5 sequences)

SEQ ID NO: 1 - DNA, 3111 bp, homo sapiens
  CDS (1)..(3108)
atggctttgcggggcgccgcgggagcgaccgacaccccggtgtcctcggccgggggagcccccggcggctcagcgtcctcgtcgtccacctcctcgggcggctcggcctcggcgggcgcggggctgtgggccgcgctctatgactacgaggctcgcggcgaggacgagctgagcctgcggcgcggccagctggtggaggtgctgtcgcaggacgccgccgtgtcgggcgacgagggctggtgggcaggccaggtgcagcggcgcctcggcatcttccccgccaactacgtggctccctgccgcccggccgccagccccgcgccgccgccctcgcggcccagctccccggtacacgtcgccttcgagcggctggagctgaaggagctcatcggcgctgggggcttcgggcaggtgtaccgcgccacctggcagggccaggaggtggccgtgaaggcggcgcgccaggacccggagcaggacgcggcggcggctgccgagagcgtgcggcgcgaggctcggctcttcgccatgctgcggcaccccaacatcatcgagctgcgcggcgtgtgcctgcagcagccgcacctctgcctggtgctggagttcgcccgcggcggagcgctcaaccgagcgctggccgctgccaacgccgccccggacccgcgcgcgcccggcccccgccgcgcgcgccgcatccctccgcacgtgctggtcaactgggccgtgcagatagcgcggggcatgctctacctgcatgaggaggccttcgtgcccatcctgcaccgggacctcaagtccagcaacattttgctacttgagaagatagaacatgatgacatctgcaataaaactttgaagattacagattttgggttggcgagggaatggcacaggaccaccaaaatgagcacagcaggcacctatgcctggatggcccccgaagtgatcaagtcttccttgttttctaagggaagcgacatctggagctatggagtgctgctgtgggaactgctcaccggagaagtcccctatcggggcattgatggcctcgccgtggcttatggggtagcagtcaataaactcactttgcccattccatccacctgccctgagccgtttgccaagctcatgaaagaatgctggcaacaagaccctcatattcgtccatcgtttgccttaattctcgaacagttgactgctattgaaggggcagtgatgactgagatgcctcaagaatcttttcattccatgcaagatgactggaaactagaaattcaacaaatgtttgatgagttgagaacaaaggaaaaggagctgcgatcccgggaagaggagctgactcgggcggctctgcagcagaagtctcaggaggagctgctaaagcggcgtgagcagcagctggcagagcgcgagatcgacgtgctggagcgggaacttaacattctgatattccagctaaaccaggagaagcccaaggtaaagaagaggaagggcaagtttaagagaagtcgtttaaagctcaaagatggacatcgaatcagtttaccttcagatttccagcacaagataaccgtgcaggcctctcccaacttggacaaacggcggagcctgaacagcagcagttccagtcccccgagcagccccacaatgatgccccgactccgagccatacagttgacttcagatgaaagcaataaaacttggggaaggaacacagtctttcgacaagaagaatttgaggatgtaaaaaggaattttaagaaaaaaggttgtacctggggaccaaattccattcaaatgaaagatagaacagattgcaaagaaaggataagacctctctccgatggcaacagtccttggtcaactatcttaataaaaaatcagaaaaccatgcccttggcttcattgtttgtggaccagccagggtcctgtgaagagccaaaactttcccctgatggattagaacacagaaaaccaaaacaaataaaattgcctagtcaggcctacattgatctacctcttgggaaagatgctcagagagagaatcctgcagaagctgaaagctgggaggaggcagcctctgcgaatgctgccacagtctccattgagatgactcctacgaatagtctgagtagatccccccagagaaagaaaacggagtcagctctgtatgggtgcaccgtccttctggcatcggtggctctgggactggacctcagagagcttcataaagcacaggctgctgaagaaccgttgcccaaggaagagaagaagaaacgagagggaatcttccagcgggcttccaagtcccgcagaagcgccagtcctcccacaagcctgccatccacctgtggggaggccagcagcccaccctccctgccactgtcaagtgccctgggcatcctctccacaccttctttctccacaaagtgcctgctgcagatggacagtgaagatccactggtggacagtgcacctgtcacttgtgactctgagatgctcactccggatttttgtcccactgccccaggaagtggtcgtgagccagccctcatgccaagacttgacactgattgtagtgtatcaagaaacttgccgtcttccttcctacagcagacatgtgggaatgtaccttactgtgcttcttcaaaacatagaccgtcacatcacagacggaccatgtctgatggaaatccgaccccaactggtgcaactattatctcagccactggagcctctgcactgccactctgcccctcacctgctcctcacagtcatctgccaagggaggtctcacccaagaagcacagcactgtccacatcgtgcctcagcgtcgccctgcctccctgagaagccgctcagatctgcctcaggcttacccacagacagcagtgtctcagctggcacagactgcctgtgtagtgggtcgcccaggaccacatcccacccaattcctcgctgccaaggagagaactaaatcccatgtgccttcattactggatgctgacgtggaaggtcagagcagggactacactgtgccactgtgcagaatgaggagcaaaaccagccggccatctatatatgaactggagaaagaattcctgtcttaa

SEQ ID NO: 2 - protein, 1036 aa, homo sapiens
MALRGAAGATDTPVSSAGGAPGGSASSSSTSSGGSASAGAGLWAALYDYEARGEDELSLRRGQLVEVLSQDAAVSGDEGWWAGQVQRRLGIFPANYVAPCRPAASPAPPPSRPSSPVHVAFERLELKELIGAGGFGQVYRATWQGQEVAVKAARQDPEQDAAAAAESVRREARLFAMLRHPNIIELRGVCLQQPHLCLVLEFARGGALNRALAAANAAPDPRAPGPRRARRIPPHVLVNWAVQIARGMLYLHEEAFVPILHRDLKSSNILLLEKIEHDDICNKTLKITDFGLAREWHRTTKMSTAGTYAWMAPEVIKSSLFSKGSDIWSYGVLLWELLTGEVPYRGIDGLAVAYGVAVNKLTLPIPSTCPEPFAKLMKECWQQDPHIRPSFALILEQLTAIEGAVMTEMPQESFHSMQDDWKLEIQQMFDELRTKEKELRSREEELTRAALQQKSQEELLKRREQQLAEREIDVLERELNILIFQLNQEKPKVKKRKGKFKRSRLKLKDGHRISLPSDFQHKITVQASPNLDKRRSLNSSSSSPPSSPTMMPRLRAIQLTSDESNKTWGRNTVFRQEEFEDVKRNFKKKGCTWGPNSIQMKDRTDCKERIRPLSDGNSPWSTILIKNQKTMPLASLFVDQPGSCEEPKLSPDGLEHRKPKQIKLPSQAYIDLPLGKDAQRENPAEAESWEEAASANAATVSIEMTPTNSLSRSPQRKKTESALYGCTVLLASVALGLDLRELHKAQAAEEPLPKEEKKKREGIFQRASKSRRSASPPTSLPSTCGEASSPPSLPLSSALGILSTPSFSTKCLLQMDSEDPLVDSAPVTCDSEMLTPDFCPTAPGSGREPALMPRLDTDCSVSRNLPSSFLQQTCGNVPYCASSKHRPSHHRRTMSDGNPTPTGATIISATGASALPLCPSPAPHSHLPREVSPKKHSTVHIVPQRRPASLRSRSDLPQAYPQTAVSQLAQTACVVGRPGPHPTQFLAAKERTKSHVPSLLDADVEGQSRDYTVPLCRMRSKTSRPSIYELEKEFLS

SEQ ID NO: 3 - protein, 8 aa, Artificial Sequence
  Synthetic polypeptide sequence
HRDLKSRN

SEQ ID NO: 4 - DNA, 28 bp, Artificial Sequence
  PCR Primers
atgaaagaatgctggcaacaagaccctc

SEQ ID NO: 5 - DNA, 28 bp, Artificial Sequence
  PCR Primers
aggtaaactgattcgatgtccatctttg